(19) United States
(12) Patent Application Publication
(10) Pub. No.: US 2009/0005459 A1
(43) Pub. Date: Jan. 1, 2009

(54) NMDA receptor antagonists and their use in inhibiting abnormal hyperphosphorylation of microtubule associated protein tau

(76) Inventors: Khalid Iqbal, Staten Island, NY (US); Inge Grundke-Iqbal, Staten Island, NY (US)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 12/151,996

(22) Filed: May 12, 2008

Related U.S. Application Data
(63) Continuation of application No. 10/622,163, filed on Jul. 17, 2003, now abandoned.
(60) Provisional application No. 60/397,434, filed on Jul. 19, 2002.

(51) Int. Cl. A61K 31/13; A61P 25/00

(52) U.S. Cl. 514/662

(57) ABSTRACT

Aminocyclohexane and aminoalkylcyclohexane compounds, which are systemically-active as NMDA receptor antagonists, are effective in inhibiting abnormal hyperphosphorylation of microtubule associated protein tau, method of treating disorders resulting from or associated with abnormal hyperphosphorylation of microtubule associated protein tau, and pharmaceutical compositions comprising the same.

### BACKGROUND OF THE INVENTION

1. Field of the Invention

Aminocyclohexane derivatives, including 1-aminoalkylcyclohexane and 1-aminoadamantane compounds, which are systemically-active as NMDA receptor antagonists, are effective in inhibiting abnormal hyperphosphorylation of microtubule associated protein tau, method of treating disorders resulting from or associated with abnormal hyperphosphorylation of microtubule associated protein tau, and pharmaceutical compositions comprising the same.

2. Description of Related Art

Neurofibrillary tangles and deposits of fibrillar amyloid beta peptides in the brain is a pathological hallmark of Alzheimer's disease (AD). Neurofibrillary tangles are inclusions located within cell bodies and proximal dendrites, and within filamentous swellings in distal axons and synaptic terminals. Hyperphosphorylated isoforms of the microtubule-associated protein tau, which assemble into poorly soluble paired helical filaments (PHF), twisted ribbons or straight filaments (SF), are a central feature of these neurofibrillary tangles (Grundke-Iqbal, et al. 1986a, 1986b; Iqbal, et al. 1986, 1989; Alonso, et al. 2001a; Goedert et al., Curr. Opin. Neurobiol., 1998, 8: 619-632). Prominent filamentous tau inclusions and brain degeneration in the absence of beta-amyloid deposits are also hallmarks of neurodegenerative tauopathies exemplified by frontotemporal dementia with parkinsonism linked to chromosome 17 (FTDP-17), corticobasal degeneration (CBD), progressive supranuclear palsy (PSP), progressive subcortical gliosis (PSG), Pick's disease (PiD), Niemann-Pick type C(NPC) neurodegenerative storage disease, as well as Argyrophilic Grain disease and correlates directly with dementia (Tomlinson, et al. 1970; Alafuzoff, et al. 1987; Arrigada, et al, 1992; Tolnay and Probst, 1999). Because multiple tau gene mutations are pathogenic for FTDP-17 and tau polymorphisms appear to be genetic risk factors for sporadic progressive supranuclear palsy and corticobasal degeneration, tau abnormalities are linked directly to the etiology and pathogenesis of various neurodegenerative diseases (Higuchi et al., Neuron, 35:433-46, 2002; Hong et al., Science, 282:1914-1917, 1998; Hutton et al., Nature, 393:702-705, 1998; Poorkaj, et al. 1998; Hutton, et al. 1998; Spillantini, et al. 1998), and may be observed before clinical onset of a neurodegenerative disease.

The biological activity of tau is regulated by its degree of phosphorylation (Lindwall and Cole, 1984). While normal tau promotes the assembly and maintains the structure of microtubules (Weingarten, et al. 1975), the abnormally hyperphosphorylated form of this protein instead sequesters normal tau, MAP1 and MAP2, binds poorly to microtubules and thereby alters the stability of microtubules and affects intracellular transport, cellular geometry, and neuronal viability (Alonso, et al. 1994, 1996, 1997; Kins et al., J. Biol. Chem., 276:38193-200, 2001). This toxic property of the AD P-tau, which through the breakdown of the microtubule network can compromise axonal transport and lead to neurodegeneration, appears to be solely due to its abnormal hyperphosphorylation because dephosphorylation by a phosphatase restores it into a normal-like protein in vitro (Alonso, et al. 1997, 2001b; Wang, et al. 1995, 1996). The abnormal hyperphosphorylation of tau in AD is believed to be due to a protein phosphorylation/dephosphorylation imbalance (Grundke-Iqbal, et al. 1986b; Iqbal, et al. 1986). To date at least 21 sites have been identified at which tau in AD brain is abnormally hyperphosphorylated (Morishima-Kawashima, et al. 1995; Iqbal and Grundke-Iqbal, 1995). About half of these sites are canonical sites for proline-directed protein kinases and tau has been found to be phosphorylated only at serines/threonines in AD. Among several different protein kinases that have been implicated in the phosphorylation of tau only the activity of cdk5 has been reported to be increased in AD brain (Patrick, et al. 1999) and even this finding was not reproduced by another laboratory (Hasagawa, et al. 2000).

On the other hand, there is accumulating evidence that reduced activities of phosphatases are also involved (Kins et al., J. Biol. Chem., 276:38193-200, 2001; Gong, et al. 2000; Bennecib, et al. 2000, 2001). The serine/threonine-specific protein phosphatases PP-2A, PP-2B, and, to a lesser extent, PP-1 were shown to efficiently dephosphorylate tau isolated from AD brain (Gong et al., FEBS Lett., 341: 94-98, 1994; Wang et al., J. Biol. Chem., 270:4854-4860, 1995). Indeed, the activity of PP-2A is decreased by about 20% in AD brain (Gong, et al. 1993, 1995). Pharmacological inhibition of PP-2A/PP-1 by okadaic acid or calyculin A induced abnormal hyperphosphorylation of tau in cultured neuroblastoma cells, metabolically active brain slices and in normal adult rats further suggesting that these phosphatases are involved in tau dephosphorylation (Tanaka, et al. 1998; Gong, et al. 2000; Bennecib, et al. 2000a, 2000b, 2001; Kins et al., J. Biol. Chem., 276:38193-200, 2001). Reduction of PP-2A activity in the rat hippocampus in vivo has been shown to produce tau hyperphosphorylation at Ser396/Ser404 and Ser262/Ser356 sites and impairment of spatial memory (Sun et al., Neuroscience 118: 1175-82, 2003). Ser-262 phosphorylated in okadaic acid-induced tau hyperphosphorylation models is one of the major sites phosphorylated in AD P-tau. This phosphorylation site is the only one that resides in the microtubule binding domains and is believed to be involved in microtubule dynamics (Biernat, et al. 1992; Singh, et al. 1996; Sironi, et al. 1998). Phosphorylation of tau at this site reduces the ability of tau to bind to microtubules and to promote their assembly (Lindwall and Cole, 1984; Singh, et al. 1996). CaM Kinase II (CaMKII) which is the most abundant of the known Ca²⁺-regulated protein kinases in the brain, is a major tau Ser-262 kinase (Sironi, et al. 1998; Bennecib, et al. 2001). A role for PP-2A in tau dephosphorylation is also supported by the finding that PP-2A is localized on microtubules and that it binds directly to tau (Sontag et al., J. Biol. Chem., 274:25490-25498, 1999). FTDP-17-associated mutations in tau induce a decrease in binding affinity for PP-2A, suggesting that altered interactions between PP2A and tau may contribute to FTDP-17 pathogenesis (Goedert et al., J. Neurochem., 75:2155-2162, 2000). The prolyl isomerase Pin1, which co-purifies with tau filament preparations, catalyzes prolyl isomerization of specific Ser/Thr-Pro motifs in tau and thereby restores the function of tau and facilitates dephosphorylation by PP-2A (Zhou et al., Mol. Cell, 6:873-883, 2000). Furthermore, in transgenic mice expressing a dominant negative mutant form of the catalytic subunit Ca of PP-2A, L199P, PP-2A activity reduces to 66% of that in wildtype littermates. In these mice, the endogenous tau is hyperphosphorylated (at Ser202/Ther205 and at Ser422 sites) and accumulated in aggregates in the somatodendritic compartments, and it is colocalized with ubiquitin reflecting an early step in the neurofibrillary lesion formation (Kins et al., J. Bio. Chem., 276: 38193-38200, 2001). Together, these data demonstrate the importance of serine/threonine-specific protein phosphatases and, in particular, PP-2A for tau function in tauopathies.

Increasing evidence supports that escalating levels of excitatory amino acids might be responsible for neuronal cell death in a variety of chronic neurodegenerative diseases including AD and other tauopathies. A predominant form of neurotoxicity appears to be mediated by excessive activation of NMDA receptor which results in calcium influx. This influx of calcium, the second messenger, activates CaMKII and regulates various functions of neurons including neurotransmitter release, synaptic plasticity and gene expression (Berridge, et al. 2000). Glutamate receptor is a known substrate of CaMKII and the phosphorylation of the glutamate receptor leads to a positive modulation of receptor function and maintenance of synaptic excitability. CaMKII activity is upregulated by glutamate and this increase in the kinase activity can be blocked by N-methyl-D-aspartate (NMDA) receptor antagonists. A recent study has shown that the NMDA receptor is in a complex with PP-2A and that stimulation of NMDA receptor can lead to the dissociation of PP-2A from the complex and the reduction of PP-2A activity (Shing, et al. 2001).

### THE PRESENT INVENTION

It has now been found that certain 1-aminocyclohexanes and 1-aminoalkylcyclohexanes possess a surprising ability to inhibit the abnormal hyperphosphorylation of microtubule associated protein tau. Thus, these substances are suited for the treatment of a wide range of CNS disorders which involve abnormal hyperphosphorylation of microtubule associated protein tau.

The 1-aminocyclohexanes are low to moderate affinity uncompetitive NMDA antagonists which can decrease neurotoxicity by inhibiting Ca²⁺ influx and have been employed for treating dementias for the last ˜ten years.

Memantine (1-amino-3,5-dimethyl adamantane) is an analog of 1-amino-cyclohexane (disclosed, e.g., in U.S. Pat. Nos. 4,122,193; 4,273,774; 5,061,703). Neramexane (1-amino-1,3,3,5,5-pentamethylcyclohexane) is also a derivative of 1-aminocyclohexane (disclosed, e.g., in U.S. Pat. No. 6,034,134). Memantine, related 1-aminoadamantane derivatives, neramexane as well as some other 1-aminoalkyl-cyclohexanes are systemically-active noncompetitive NMDA receptor antagonists having moderate affinity for the receptor. They exhibit strong voltage dependent characteristics and fast blocking/unblocking kinetics (Parsons et al., 1999, supra; Görtelmeyer et al., Arzneim-Forsch/Drug Res., 1992, 42:904-913; Winblad et al., Int. J. Geriat. Psychiatry, 1999, 14:135-146; Rogawski, Amino Acids, 2000, 19: 133-49; Danysz et al., Curr. Pharm. Des., 2002, 8:835-43; Jirgensons et. al., Eur. J. Med. Chem., 2000, 35: 555-565). These compounds dissociate from the NMDA receptor channels much more rapidly than the high affinity NMDA receptor antagonists such as (+)MK-801 and attenuate disruption of neuronal plasticity produced by tonic overstimulation of NMDA receptors probably by causing an increase of the signal-to-noise ratio. Due to their relatively low affinity for the receptor, strong voltage dependency and fast receptor unblocking kinetics, these compounds are essentially devoid of the side effects of other NMDA receptor antagonists at doses within the therapeutic range (Kornhuber et al., Eur. J. Pharmacol., 1991, 206:297-311). Indeed, memantine has been applied clinically for over 15 years showing good tolerability with the number of treated patients exceeding 200,000 (Parsons et al., 1999, supra).

Memantine, neramexane as well as other 1-aminoalkylcyclohexanes (many of which are actually 1-aminoadamantane derivatives) have been suggested to be useful in alleviation of various progressive neurodegenerative disorders such as dementia in AD, Parkinson's disease, and spasticity (see, e.g., U.S. Pat. Nos. 5,061,703; 5,614,560, and 6,034,134; Parsons et al., 1999, supra; Möbius, ADAD, 1999, 13:S172-178; Danysz et al., Neurotox. Res., 2000, 2:85-97; Winblad and Poritis, Int. J. Geriatr. Psychiatry, 1999, 14:135-146; Görtelmeyer et al., 1992, supra; Danysz et al., Curr. Pharm. Des., 2002′, 8:835-843; Jirgensons et. al., Eur. J. Med. Chem., 2000, 35: 555-565). Chronic treatment of adult rats with memantine has been shown to enhance the formation of hippocampal long-term potentiation, increase the durability of synaptic plasticity, improve spatial memory abilities, and reverse the memory impairment produced by NMDA receptor agonists (Barnes et al., Eur. J. Neurosci., 1996; 8:65-571; Zajaczkowski et al., Neuropharm, 1997, 36:961-971). Treatment with Memantine leads to functional improvement and reduces care dependence in severely demented patients (Winblad, et al. 1999). Several preclinical studies have indicated that therapeutic concentrations of Memantine might be neuroprotective, especially in chronic neurodegenerative disease, such as AD, without producing side effects such as impairment of learning and long term potentiation (LTP) or induction of pyschotomimetic-like behavioral syndromes (Muller, et al. 1995; Danysa, et al. 1997; Parsons, et al. 1999).

The present invention is based on the inventors' discovery that Memantine decreases the abnormal hyperphosphorylation of tau and the relative activity of tau kinases and phosphatases in organotypic culture of adult rat hippocampal slices in which PP-2A activity was inhibited by okadaic acid. The inventors find that (i) Memantine restores the okadaic acid-induced increase in CaMKII and decrease in PP-2A activities and abnormal hyperphosphorylation of tau to the control level; and (ii) that Memantine reverses the expression and aggregation of microtubule associated protein 2 (MAP2) and the phosphorylation and aggregation of neurofilament heavy and medium (NF-H/M) subunits.

Despite abundant data on their clinical effects, the ability of NMDA inhibitors to affect directly the abnormal hyperphosphorylation of tau and the relative activity of tau kinases and phosphatases has not been suggested. Also, there is clearly a need in the art for a more effective treatment of mammals suffering from tauopathies. The present inventors have satisfied this need by conceiving and demonstrating for the first time that NMDA receptor antagonists such as 1-aminocyclohexane derivatives (e.g., memantine or neramexane) are able to decrease the abnormal hyperphosphorylation of tau and may be utilized for treatment of a broad range of neurodegenerative disorders.

Moreover, the applicants have compared the activity of Memantine to restore okadaic acid-induced inhibition of PP-2A activity with two known NMDA receptor antagonists, D-(−)-2-amino-5-phospho-pentanoic acid (AP) and 5,7-dichlorokynurenic acid (DK). The findings demonstrate that restoration of the PP-2A activity and phosphorylation of tau at Ser-262 by 5 μM Memantine is apparently independent of its activity as an NMDA receptor antagonist because similar effects were not observed with 5 μM AP or 5 μM DK. Thus, it may be concluded that Memantine inhibits abnormal hyperphosphorylation of tau by mediating PP-2A signaling.

### OBJECTS OF THE INVENTION

It is an object of the present invention to provide novel pharmaceutical compounds which are aminocyclohexane and aminoalkylcyclohexane NMDA receptor antagonists, which compounds function to inhibit abnormal hyperphosphorylation of microtubule associated protein tau, and pharmaceutical compositions thereof. It is a further object of the invention to provide a novel method of treating, eliminating, alleviating, palliating, or ameliorating undesirable neurodegenerative CNS disorders which involve disturbances of phosphorylation of microtubule associated protein tau.

Yet additional objects will become apparent hereinafter, and still further objects will be apparent to one skilled in the art.

### SUMMARY OF THE INVENTION

What we therefore believe to be comprised by our invention may be summarized inter alia in the following words:

A method for the prevention, treatment or relief of a state, disorder or condition resulting from hyperphosphorylation of microtubule protein tau, which method is useful for: (1) preventing or delaying the appearance of clinical symptoms and parameters such as neurodegeneration of the state, disorder or condition developing in a mammal that may be afflicted with or predisposed to the state, disorder or condition but does not yet experience or display clinical symptoms and parameters of the state, disorder or condition, (2) inhibiting the state, disorder or condition, i.e., arresting or reducing the development of the disease or at least one clinical symptom thereof, or (3) relieving the disease, i.e., causing regression of the state, disorder or condition or at least one of its clinical symptoms and parameters, such method comprising the step of administering, to a patient in need thereof, an effective amount of an aminocyclohexane or an aminoalkylcyclohexane, preferably memantine or neramexane.

A method for the prevention, treatment or relief of a state, disorder or condition resulting from hyperphosphorylation of microtubule protein tau, which method is useful for: (1) preventing or delaying the appearance of clinical symptoms and parameters such as neurodegeneration of the state, disorder or condition developing in a mammal that may be afflicted with or predisposed to the state, disorder or condition but does not yet experience or display clinical symptoms and parameters of the state, disorder or condition, (2) inhibiting the state, disorder or condition, i.e., arresting or reducing the development of the disease or at least one clinical symptom thereof, or (3) relieving the disease, i.e., causing regression of the state, disorder or condition or at least one of its clinical symptoms and parameters, such method comprising the step of administering, to a patient in need thereof, an effective amount of a compound selected from those of formula I:

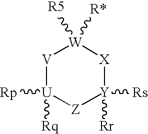

wherein:
R* is -(A)ₙ-(CR¹R²)ₘ—NR³R⁴,
n+m=0, 1, or 2,
A is selected from the group linear or branched lower alkyl (C₁-C₆), linear or branched lower alkenyl (C₂-C₆), and linear or branched lower alkynyl (C₂-C₆),
R¹ and R² are independently selected from the group hydrogen, linear or branched lower alkyl (C₁-C₆), linear or branched lower alkenyl (C₂-C₆), and linear or branched lower alkynyl (C₂-C₆),
R³ and R⁴ are independently selected from the group hydrogen, linear or branched lower alkyl (C₁-C₆), linear or branched lower alkenyl (C₂-C₆), and linear or branched lower alkynyl (C₂-C₆), or together form alkylene (C₂-C₁₀) or alkenylene (C₂-C₁₀) or together with the N form a 3-7-membered azacycloalkane or azacycloalkene, including substituted (alkyl (C₁-C₆), alkenyl (C₂-C₆)) 3-7-membered azacycloalkane or azacycloalkene,
R⁵ is independently selected from the group hydrogen, linear or branched lower alkyl (C₁-C₆), linear or branched lower alkenyl (C₂-C₆), and linear or branched lower alkynyl (C₂-C₆), or R⁵ combines with the carbon to which it is attached and the next adjacent ring carbon to form a double bond,
Rₚ, R_{q}, Rᵣ, and Rₛ are independently selected from the group hydrogen, linear or branched lower alkyl (C₁-C₆), linear or branched lower alkenyl (C₂-C₆), linear or branched lower alkynyl (C₂-C₆), cycloalkyl (C₃-C₆) and phenyl, or Rₚ, R_{q}, Rᵣ, and Rₛ independently may combine with the carbon to which it is attached and the next adjacent carbon to form a double bond, or Rₚ, R_{q}, Rᵣ, and Rₛ may combine together to represent lower alkylene —(CH₂)ₓ— bridge wherein x is 2-5, inclusive, which alkylene bridge may, in turn, combine with R⁵ to form a additional lower alkylene —(CH₂)_{y}— bridge, wherein y is 1-3, inclusive,
U-V-W-X-Y-Z is selected from cyclohexane,
   cyclohex-2-ene,
   cyclohex-3-ene,
   cyclohex-1,4-diene,
   cyclohex-1,5-diene,
   cyclohex-2,4-diene, and
   cyclohex-2,5-diene,
      and its optical isomers and pharmaceutically-acceptable acid or base addition salt thereof.

A method for the prevention, treatment or relief of a state, disorder or condition resulting from hyperphosphorylation of microtubule protein tau, which method is useful for: (1) preventing or delaying the appearance of clinical symptoms and parameters of the state, disorder or condition developing in a mammal that may be afflicted with or predisposed to the state, disorder or condition but does not yet experience or display clinical symptoms and parameters of the state, disorder or condition, (2) inhibiting the state, disorder or condition, i.e., arresting or reducing the development of the disease or at least one clinical symptom thereof, or (3) relieving the disease, i.e., causing regression of the state, disorder or condition or at least one of its clinical symptoms and parameters, such method comprising the step of administering, to a patient in need thereof, an effective amount of an aminocyclohexane.

A method for the prevention, treatment or relief of a state, disorder or condition resulting from hyperphosphorylation of microtubule protein tau, which method is useful for: (1) preventing or delaying the appearance of clinical symptoms and parameters such as neurodegeneration of the state, disorder or condition developing in a mammal that may be afflicted with or predisposed to the state, disorder or condition but does not yet experience or display clinical symptoms and parameters of the state, disorder or condition, (2) inhibiting the state, disorder or condition, i.e., arresting or reducing the development of the disease or at least one clinical symptom thereof, or (3) relieving the disease, i.e., causing regression of the state, disorder or condition or at least one of its clinical symptoms and parameters, such method comprising the step of administering, to a patient in need thereof, an effective amount of an aminocyclohexane selected from the group:
1-amino adamantane,
1-amino-3-phenyl adamantane,
1-amino-methyl-adamantane,
1-amino-3,5-dimethyl adamantane,
1-amino-3-ethyl adamantane,
1-amino-3-isopropyl adamantane,
1-amino-3-n-butyl adamantane,
1-amino-3,5-diethyl adamantane,
1-amino-3,5-diisopropyl adamantane,
1-amino-3,5-di-n-butyl adamantane,
1-amino-3-methyl-5-ethyl adamantane,
1-N-methylamino-3,5-dimethyl adamantane,
1-N-ethylamino-3,5-dimethyl adamantane,
1-N-isopropyl-amino-3,5-dimethyl adamantane,
1-N,N-dimethyl-amino-3,5-dimethyl adamantane,
1-N-methyl-N-isopropyl-amino-3-methyl-5-ethyl adamantane,
1-amino-3-butyl-5-phenyl adamantane,
1-amino-3-pentyl adamantane,
1-amino-3,5-dipentyl adamantane,
1-amino-3-pentyl-5-hexyl adamantane,
1-amino-3-pentyl-5-cyclohexyl adamantane,
1-amino-3-pentyl-5-phenyl adamantane,
1-amino-3-hexyl adamantane,
1-amino-3,5-dihexyl adamantane,
1-amino-3-hexyl-5-cyclohexyl adamantane,
1-amino-3-hexyl-5-phenyl adamantane,
1-amino-3-cyclohexyl adamantane,
1-amino-3,5-dicyclohexyl adamantane,
1-amino-3-cyclohexyl-5-phenyl adamantane,
1-amino-3,5-diphenyl adamantane,
1-amino-3,5,7-trimethyl adamantane,
1-amino-3,5-dimethyl-7-ethyl adamantane,
1-amino-3,5-diethyl-7-methyl adamantane,
1-amino-3-methyl-5-propyl adamantane,
1-amino-3-methyl-5-butyl adamantane,
1-amino-3-methyl-5-pentyl adamantane,
1-amino-3-methyl-5-hexyl adamantane,
1-amino-3-methyl-5-cyclohexyl adamantane,
1-amino-3-methyl-5-phenyl adamantane,
1-amino-3-ethyl-5-propyl adamantane,
1-amino-3-ethyl-5-butyl adamantane,
1-amino-3-ethyl-5-pentyl adamantane,
1-amino-3-ethyl-5-hexyl adamantane,
1-amino-3-ethyl-5-cyclohexyl adamantane,
1-amino-3-ethyl-5-phenyl adamantane,
1-amino-3-propyl-5-butyl adamantane,
1-amino-3-propyl-5-pentyl adamantane,
1-amino-3-propyl-5-hexyl adamantane,
1-amino-3-propyl-5-cyclohexyl adamantane,
1-amino-3-propyl-5-phenyl adamantane,
1-amino-3-butyl-5-pentyl adamantane,
1-amino-3-butyl-5-hexyl adamantane,
1-amino-3-butyl-5-cyclohexyl adamantane,

and their acid addition compounds.

A method for the prevention, treatment or relief of a state, disorder or condition resulting from hyperphosphorylation of microtubule protein tau, which method is useful for: (1) preventing or delaying the appearance of clinical symptoms and parameters of the state, disorder or condition developing in a mammal that may be afflicted with or predisposed to the state, disorder or condition but does not yet experience or display clinical symptoms and parameters of the state, disorder or condition, (2) inhibiting the state, disorder or condition, i.e., arresting or reducing the development of the disease or at least one clinical symptom thereof, or (3) relieving the disease, i.e., causing regression of the state, disorder or condition or at least one of its clinical symptoms and parameters, such method comprising the step of administering, to a patient in need thereof, an effective amount of an aminocyclohexane which is an aminoalkylcyclohexane.

A method for the prevention, treatment or relief of a state, disorder or condition resulting from hyperphosphorylation of microtubule protein tau, which method is useful for: (1) preventing or delaying the appearance of clinical symptoms and parameters such as neurodegeneration of the state of the state, disorder or condition developing in a mammal that may be afflicted with or predisposed to the state, disorder or condition but does not yet experience or display clinical symptoms and parameters of the state, disorder or condition, (2) inhibiting the state, disorder or condition, i.e., arresting or reducing the development of the disease or at least one clinical symptom thereof, or (3) relieving the disease, i.e., causing regression of the state, disorder or condition or at least one of its clinical symptoms and parameters, such method comprising the step of administering, to a patient in need thereof, an effective amount of an amino-alkylcyclohexane selected from the group:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane,
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane, N-ethyl-1-amino-1,3,3,5,5-pentamethylcyclohexane, and N-(1,3,3,5,5-pentamethylcyclohexyl)pyrrolidine,
   and their acid addition compounds.

A method for the prevention, treatment or relief of a state, disorder or condition resulting from hyperphosphorylation of microtubule protein tau, which method is useful for: (1) preventing or delaying the appearance of clinical symptoms and parameters such as neurodegeneration of the state of the state, disorder or condition developing in a mammal that may be afflicted with or predisposed to the state, disorder or condition but does not yet experience or display clinical symptoms and parameters of the state, disorder or condition, (2) inhibiting the state, disorder or condition, i.e., arresting or reducing the development of the disease or at least one clinical symptom thereof, or (3) relieving the disease, i.e., causing regression of the state, disorder or condition or at least one of its clinical symptoms and parameters, wherein such state, disorder, or condition results from hyperphosphorylation of microtubule protein tau, wherein the state, disorder or condition causes neurofibrillary tangles, neuropile threads, dystrophic neruites of neuritic plaques, or Pick bodies, such method comprising the step of administering, to a patient in need thereof, an effective amount of an aminocyclohexane or an aminoalkylcyclohexane, preferably memantine or neramexane.

A method for the prevention, treatment or relief of a state, disorder or condition resulting from hyperphosphorylation of microtubule protein tau, which method is useful for: (1) preventing or delaying the appearance of clinical symptoms and parameters of the state, disorder or condition developing in a mammal that may be afflicted with or predisposed to the state, disorder or condition but does not yet experience or display clinical symptoms and parameters of the state, disorder or condition, (2) inhibiting the state, disorder or condition, i.e., arresting or reducing the development of the disease or at least one clinical symptom thereof, or (3) relieving the disease, i.e., causing regression of the state, disorder or condition or at least one of its clinical symptoms and parameters, wherein such state, disorder, or condition results from hyperphosphorylation of microtubule protein tau, and wherein the state, disorder or condition is selected from the group: amyotrophic lateral sclerosis, parkinsonism-dementia, argyrophilic grain dementia, British type amyloid angiopathy, corticobasal degeneration, dementia pugilistica, autism with self-injury behavior, Down's syndrome, FTDP-17, Gerstmann-Straussler-Scheinker disease, Hallenvorden-Spatz disease, inclusion body myositis, multiple system atrophy, myotonic dystrophy, Niemann-Pick disease type C, Pick's disease, presenile dementia, prion protein cerebral amyloid angiopathy, progressive supranuclear palsy, progressive subcortical gliosis, post-encephalitic parkinsonism, subacute sclerosing panencephalitis, tangle only dementia, dementia in Alzheimer's Disease, Parkinson's disease, spasticity, AIDS dementia, neuropathic pain, cerebral ischemia, epilepsy, glaucoma, hepatic encephalopathy, multiple sclerosis, stroke, tardive dyskinesia, drug tolerance, opiate/alcohol dependence, thermal hyperalgesia, mechanical allodynia, and may also possess immunomodulatory, antimalarial, anti-Borna virus, and anti-Hepatitis C activities, such method comprising the step of administering, to a patient in need thereof, an effective amount of an aminocyclohexane or an aminoalkylcyclohexane, preferably memantine or neramexane.

A method for decreasing the abnormal hyperphosphorylation of microtubule protein tau in a mammal, such method comprising administering to said mammal an effective amount of an aminocyclohexane or an aminoalkylcyclohexane, preferably memantine or neramexane.

A method for decreasing neurofibrillary tangles, neuropile threads, dystrophic neruites of neuritic plaques, or Pick bodies in a mammal, such method comprising administering to said mammal an effective amount of memantine or neramexane.

Moreover, the applicants have compared the activity of Memantine to restore okadaic acid-induced inhibition of PP-2A activity with two known NMDA receptor antagonists, D-(−)-2-amino-5-phospho-pentanoic acid (AP) and 5,7-dichlorokynurenic acid (DK). The findings demonstrate that restoration of the PP-2A activity and phosphorylation of tau at Ser-262 by 5 μM Memantine is apparently independent of its activity as an NMDA receptor antagonist because similar effects were not observed with 5 μM AP or 5 μM DK. Thus, it may be concluded that Memantine inhibits abnormal hyperphosphorylation of tau by mediating PP-2A signaling.

### BRIEF DESCRIPTION OF THE DRAWINGS

FIG. 1. Inhibition of PP-2A and stimulation of CaMKII activities, and release of LDH (cell death) by OA in hippocampal slices in culture. Hippocampal slices were treated with either medium alone as control or with 10 nM, 100 nM or 1000 nM OA, for 3 h, 24 h or 48 h. The slices were then homogenized and centrifuged at 16000×g for 15 min and the extracts were used for assaying PP-2A, PP-1, PKA, GSK-3, cdk5 and CaMKII activities. The phosphatase and CaMKII activities were expressed as the percentage of the activity of control samples incubated in the cultured medium alone. Bars represent means±SD obtained from at least 3 independent assays. a. PP-2A activity as % of control-treated slices. A decrease of 42% (p<0.05) and 78% (p<0.01) in PP-2A activity was observed in slices treated with 100 nM OA for 24 h and with 1000 nM OA for 48 h, respectively. CaMKII activity increased to 180% (p<0.01) and 240% (p<0.01) in hippocampal slices treated for 24 h with 100 nM and 1000 nM OA, respectively. The cell death as assayed by LDH activity released in the medium (ratio of LDH activity before/after OA treatment) increased with increase in OA concentration and treatment period (p<0.001). Not shown in this figure, no significant changes in the activities of PP-1, PKA, GSK-3 or cdk5 were detected.

FIG. 2. Restoration of activities of PP-2A and CaMKII to normal level and inhibition of cell death by Memantine in OA-treated hippocampal slices. Hippocampal slices in culture were incubated either in medium alone as control or in the presence of 100 nM OA and 0, 1, 10, or 30 μM Memantine (Mem) for 3 h, 24 h or 48 h. The cell death was measured by assaying LDH activity released in the medium. The slices were then homogenized and centrifuged at 16,000×g for 15 min, and the extracts were used for assaying PP-2A, CaMKII, PKA, cdk5 and GSK-3 activities. The phosphatase and the kinase activities were expressed as the percentage of the corresponding activities of slices treated with medium alone in culture. Bars represent means±SD obtained from at least 3 independent assays. a. Memantine restored the OA-inhibited PP-2A activity to normal level (p<0.02) but had no effect on the enzyme activity in control slices. Memantine, 10 μM, during 24 h practically completely restored PP-2A activity to normal level. b. Memantine restored the OA-stimulated CaMKII activity to normal level (p<0.02). Memantine had no significant effect on activity in normal control hippocampal slices. c. As low as 1 μM Memantine restored the OA-induced increase in PKA activity to normal level (p<0.05). Not shown in this figure neither OA nor Memantine had any significant effect on either GSK-3 or cdk5 activity. d. As low as 1 μM Memantine completely inhibited the OA-induced cell death (p<0.001).

FIG. 3. Restoration of OA-induced tau phosphorylation at Ser 262 to normal level by Memantine. a. Homogenates (4 μg of protein per dot) of cultured slices after different treatments were subjected to radioimmuno-dot-blots probed with different tau antibodies and ¹²⁵I-conjugated anti-mouse/rabbit IgG as a secondary antibody. The immunoreactivities at different sites obtained with different antibodies were quantitated by a phosphorimager and then normalized against the level of total tau similarly detected with pAb R134d. b. ¹²⁵I-Western blots of the cultured hippocampal slices. Homogenates (30 μg of protein per lane) of cultured slices after different treatments were subjected to Western blots developed with pAb pS-262: 1. control, 2. 100 nM OA 24 h, then medium 24 h, 3. 100 nM OA 24 h, then 10 μM Memantine 24 h. c. Effect of different concentrations of Memantine on the restoration of the OA-induced phosphorylation of tau at Ser-262 as determined by radioimmunodot-blots as in FIG. 3a. The data are the averages of two independent assays.

d. Immunohistochemical staining showing tau phosphorylation at Ser 262 in cultured hippocampal slices. i, ii, iii, v: slices in culture treated with 100 nM OA 24 h, then medium 24 h; iv: Control, with medium only; vi: 100 nM OA 24 h, then 10 μM Memantine 24 h.
(i) Low magnification micrograph showing the distribution of tau phosphorylated at Ser-262 in a whole hippocampal slice. (ii), (iii) High magnifications of area boxed in (i) showing strongly immunopositive cells and long processes with small aggregates of phosphorylated tau (arrowheads). In control-treated slices, in the area corresponding to stratum oriens and alveus most cells were only weakly stained (iv), whereas in the OA-treated slices the number of pS262 positive cells was increased markedly in this area (v). The inset (v-i) shows pS262 positive axons with uneven contour and protein accumulations in clumps, passing through the whole width of the stratum radiatum. (vi), The number of pS262 positive cells decreased dramatically in the slices treated with 100 nM OA for 24 h followed by 10 μM Memantine for 24 h. (iv, v and vi). Same magnification.

FIG. 4. Reversal of OA-induced changes in MAP2 and neurofilaments by Memantine. a. (i-vi) Immunohistochemical staining of MAP2 (i-iii) and neurofilaments (iv-iv) in cultured hippocampal slices. i, iv: Control-treated; ii, v: Treated with 100 nM OA for 24 h, followed by medium for 24 h; iii, vi: 100 nM OA 24 h, followed by 10 μM Memantine, 24 h. (ii) In OA-treated slices MAP2 (SMI 52) immunostaining decreased and protein accumulations as clumps were visible in the dendrites of small neurons in the area corresponding to stratum oriens and alveus. (iii) in Memantine-treated slices the beaded accumulation of MAP2 in dendrites was reduced and overall staining was increased. (v) Neurofilament (SMI 31) immunostaining was increased, especially in thick, tortuous, thread-like and beaded/fragmented dendrites in the OA treated slices. (vi) Memantine reduced these changes. b, c: [¹²⁵I] Western blots showing changes in MAP2 (b) and neurofilaments (c). Homogenates (30 μg of protein per lane) of slices after different treatments in culture were subjected to Western blots developed with mAb SMI 52 to MAP2 or mAb SMI 31 to NF-H/M. Consistent with the immunohistochemical staining, the Western blots revealed that Memantine treatment increased MAP2, and reversed the OA-induced increase in phosphorylated NF-H/M.

FIG. 5. Memantine restored the activities of PP-2A and CaMKII of OA-treated hippocampal slices both when employed along with OA or followed by OA treatment. Hippocampal slices in culture were incubated in medium alone as controls or in the presence of 100 nM OA or 100 nM OA plus 10 μM Memantine for 24 h. The slices were then washed to remove OA and incubated in either medium or 10 μM Memantine for another 24 h, followed by homogenization and centrifugation at 16000×g for 15 min. The extracts were then used for assaying PP-2A and CaMKII activities. The phosphatase and kinase activities of OA or Memantine-treated samples were expressed as the percentage of the corresponding activities of control samples incubated in medium alone. Bars represent means±SD obtained from at least 3 independent assays. a. Restoration of PP-2A activity by Memantine. Memantine restored the PP-2A activity to normal level both when the tissue slices were treated with OA plus Memantine or with OA and then with Memantine (p<0.05). b. Restoration of CaMKII activity by Memantine (p<0.05). All treatments were the same as in FIG. 5a. Compare bars 4 with 5 and 4/5 with 2/3. c,d. The restoration of PP-2A activity by Memantine in hippocampal slices in culture is not due to any direct interaction between Memantine and OA. c. Addition of OA (100 nM) to the hippocampal slices extract (16000×g for 15 min) resulted in 90% inhibition of PP-2A activity, and further addition of 1, 5, 10, 30 or 60 μM Memantine to this extract had no significant effect on the phosphatase activity. d. Addition of 1, 5 or 10 μM Memantine to the 16,000×g extract of cultured hippocampal slices in which PP-2A activity had been inhibited (˜40%) by OA for 24 h, had no significant effect on the phosphatase activity (compare bars 3-5 with bars 1 and 2).

FIG. 6. Effect of glutamate on phosphorylation of tau at Ser-262 and on protein phosphatase and kinase activities in hippocampal slices in culture. Hippocampal slices in culture were first treated with 55 mM KCl, 10 min, to deblock calcium channels and then with 0.3 mM glutamate 1 h, followed by medium, 10 μM Memantine or 15 μM MK801 for 3 h, 8 h, or 24 h. The slices were then homogenized and either employed for [¹²⁵I] Western blots developed with PSer-262 tau antibody (a) or centrifuged at 16,000×g for 15 min, and the extracts used for assaying the activities of PP-2A, CaMKII and MAPK (b-d). The phosphatase and kinase activities were expressed as percentage of the activity of control samples incubated in medium alone. Bars represent means±SD obtained from at least three independent assays. 1. in medium, 3 h; 2. 0.3 mM glutamate, 1 h; 3. 0.3 mM glutamate, 1 h, followed by medium 3 h; 4. 0.3 mM glutamate, 1 h, followed by Memantine, 3 h; 5. 0.3 mM glutamate, 1 h, followed by MK801, 3 h. b. After 1 h glutamate treatment, CaMKII activity increased to ˜180% (p<0.001) and the phosphorylation of tau at Ser-262 increased markedly. But this stimulation was restored to normal level 3-8 h after the removal of glutamate. Glutamate treatment did not induce any detectable change in either MAPK activity (c) or in PP-2A activity (d).

### DETAILED DESCRIPTION OF THE INVENTION

The Okadaic Acid (OA) or calyculin A induced decrease in PP-2A activity and increase in abnormal hyperphosphorylation of tau and consequent neurodegeneration in hippocampal slices in culture is a promising ex vivo model of tauopathies/neurofibrillary degeneration. The 1-aminocyclohexanes, and particularly Memantine, modulate PP-2A signaling and inhibit neurofibrillary degeneration in this model. This activity of Memantine makes it a promising pharmacological therapeutic drug for tauopathies. For examples, the therapeutic effect of Memantine in the moderate to severe cases of AD, reported previously, might involve Memantine's action as a PP-2A signaling modulator.

Discoveries of the abnormal hyperphosphorylation of tau, the abnormal tau as the major protein subunit of PHF and the cosegregation of certain mutations in tau gene with the disease in the FTDP-17, combined with the fact that neurofibrillary degeneration is apparently required for the clinical expression of the disease in AD patients constitute an overwhelming case for the inhibition of neurofibrillary degeneration as one of the most promising therapeutic targets for AD and related tauopathies. Both in vitro and in situ data have revealed that the abnormal hyperphosphorylation converts tau into a toxic molecule where not only does it lose its ability to promote assembly and stabilize microtubules but instead it sequesters normal tau, MAP1 and MAP2, causing inhibition of assembly and disruption of microtubules, and ultimately the abnormal tau self assembles into tangles of PHF/SF (Alonso, et al., 1994, 1996, 1997, 2001a).

The phosphorylation state of a phosphoprotein is a function of a balance between the activities of the phosphoprotein phosphatases and the protein kinases to which the protein is a substrate. This balance is apparently tilted in favor of hyperphosphorylation in neurons with neurofibrillary degeneration. To date, of all the protein kinases and phosphoprotein phosphatases implicated in AD neurofibrillary degeneration, overwhelming evidence has accumulated that suggests that PP-2A is a major regulator of the phosphorylation of tau and the activity of this enzyme is compromised in AD brain (Gong, et al. 1993, 1995, 2000; Bennecib, et al. 2000, 2001).

Thus, through restoration of the PP-2A activity the abnormal hyperphosphorylation of tau and the consequent neurofibrillary degeneration might be inhibited. As specified in Examples, infra, the present inventors have shown for the first time that Memantine, an aminocyclohexane and an NMDA antagonist, can reverse the OA-induced protein phosphorylation/dephosphorylation imbalance. Furthermore, the restoration to normal state of the OA-induced reduction of the PP-2A activity and of the associated increase in the activity of CaMKII results in inhibition of the hyperphosphorylation and the aggregation of tau and NF-H/M and loss of MAP2.

OA is an extensively studied experimental irreversible inhibitor of PP-2A and PP-1 with in vitro IC₅₀ of ˜1 nM and 0.1 to 0.5 μM, respectively (Bialojan and Takai, 1988). Whereas, in previous studies the treatment of the SY5Y human neuroblastoma in culture with 10 nM OA for 24 h was found to result in a complete inhibition of PP-2A and ˜65% inhibition of PP-1, in metabolically active rat brain slices a maximal of ˜70% inhibition of only PP-2A and no detectable inhibition of PP-1 were observed with up to 5 μM of the drug during 3 h treatment (Gong, et al. 2000; Bennecib, et al. 2001).

The following details and detailed Examples are given by way of illustration only, and are not to be construed as limiting.

### Method of Treating

Due to their high degree of activity and their low toxicity, together presenting a most favorable therapeutic index, the active principles of the invention may be administered to a subject, e.g., a living animal (including a human) body, in need thereof, for the treatment, alleviation, or amelioration, palliation, or elimination of an indication or condition which is susceptible thereto, or representatively of an indication or condition set forth elsewhere in this application, preferably concurrently, simultaneously, or together with one or more pharmaceutically-acceptable excipients, carriers, or diluents, especially and preferably in the form of a pharmaceutical composition thereof, whether by oral, rectal, or parenteral (including intravenous, subcutaneous and intranasal) or in some cases even topical route, in an effective amount. Suitable dosage ranges are 1-1000 milligrams daily, preferably 10-500 milligrams daily, and especially 50-500 milligrams daily, depending as usual upon the exact mode of administration, form in which administered, the indication toward which the administration is directed, the subject involved and the body weight of the subject involved, and the preference and experience of the physician or veterinarian in charge. Treatment may be continued as long as benefits persist.

The terms aminocyclohexane and aminocyclohexane derivatives used herein is meant to describe compounds which are derived from amantadine and may include, but are not limited to, the following compounds:
1-amino adamantane,
1-amino-3-phenyl adamantane,
1-amino-methyl-adamantane,
1-amino-3,5-dimethyl adamantane,
1-amino-3-ethyl adamantane,
1-amino-3-isopropyl adamantane,
1-amino-3-n-butyl adamantane,
1-amino-3,5-diethyl adamantane,
1-amino-3,5-diisopropyl adamantane,
1-amino-3,5-di-n-butyl adamantane,
1-amino-3-methyl-5-ethyl adamantane,
1-N-methylamino-3,5-dimethyl adamantane,
1-N-ethylamino-3,5-dimethyl adamantane,
1-N-isopropyl-amino-3,5-dimethyl adamantane,
1-N,N-dimethyl-amino-3,5-dimethyl adamantane,
1-N-methyl-N-isopropyl-amino-3-methyl-5-ethyl adamantane,
1-amino-3-butyl-5-phenyl adamantane,
1-amino-3-pentyl adamantane,
1-amino-3,5-dipentyl adamantane,
1-amino-3-pentyl-5-hexyl adamantane,
1-amino-3-pentyl-5-cyclohexyl adamantane,
1-amino-3-pentyl-5-phenyl adamantane,
1-amino-3-hexyl adamantane,
1-amino-3,5-dihexyl adamantane,
1-amino-3-hexyl-5-cyclohexyl adamantane,
1-amino-3-hexyl-5-phenyl adamantane,
1-amino-3-cyclohexyl adamantane,
1-amino-3,5-dicyclohexyl adamantane,
1-amino-3-cyclohexyl-5-phenyl adamantane,
1-amino-3,5-diphenyl adamantane,
1-amino-3,5,7-trimethyl adamantane,
1-amino-3,5-dimethyl-7-ethyl adamantane,
1-amino-3,5-diethyl-7-methyl adamantane,
1-amino-3-methyl-5-propyl adamantane,
1-amino-3-methyl-5-butyl adamantane,
1-amino-3-methyl-5-pentyl adamantane,
1-amino-3-methyl-5-hexyl adamantane,
1-amino-3-methyl-5-cyclohexyl adamantane,
1-amino-3-methyl-5-phenyl adamantane,
1-amino-3-ethyl-5-propyl adamantane,
1-amino-3-ethyl-5-butyl adamantane,
1-amino-3-ethyl-5-pentyl adamantane,
1-amino-3-ethyl-5-hexyl adamantane,
1-amino-3-ethyl-5-cyclohexyl adamantane,
1-amino-3-ethyl-5-phenyl adamantane,
1-amino-3-propyl-5-butyl adamantane,
1-amino-3-propyl-5-pentyl adamantane,
1-amino-3-propyl-5-hexyl adamantane,
1-amino-3-propyl-5-cyclohexyl adamantane,
1-amino-3-propyl-5-phenyl adamantane,
1-amino-3-butyl-5-pentyl adamantane,
1-amino-3-butyl-5-hexyl adamantane,
1-amino-3-butyl-5-cyclohexyl adamantane,

and their acid addition compounds.

The terms adamantane derivatives which are aminoalkylcyclohexane used herein is meant to describe adamantane compounds which may include, but are not limited to, the following compounds:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane,
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane, N-ethyl-1-amino-1,3,3,5,5-pentamethylcyclohexane, and N-(1,3,3,5,5-pentamethylcyclohexyl)pyrrolidine, and their acid addition compounds.

### PHARMACOLOGY—SUMMARY

The active principles of the present invention, and pharmaceutical compositions thereof and method of treating therewith, are characterized by unique advantageous and unpredictable properties, rendering the “subject matter as a whole”, as claimed herein, unobvious. The compounds and pharmaceutical compositions thereof have exhibited, in standard accepted reliable test procedures, the following valuable properties and characteristics:

The active principles of the present invention are systemically-active, and (i) function to restore the abnormal (e.g., okadaic acid-induced) increase in CaMKII and decrease in PP-2A activities and abnormal hyperphosphorylation of tau to the control level; and (ii) that reverse the expression and aggregation of microtubule associated protein 2 (MAP2) and/or hyperphosphorylation and aggregation of neurofilament heavy and medium (NF-H/M) subunit; accordingly, these compounds may be of utility in the treatment, elimination, palliation, alleviation, and amelioration of responsive conditions, by application or administration to the living animal host for the treatment of a wide range of CNS disorders which involve abnormal hyperphosphorylation of microtubule associated protein tau.

### Methods

### Adult Hippocampal Organotypic Cultures

Organotypic cultures of rat hippocampal slices were prepared from 20-30 day old Wistar rats and cultured with the interface method as described previously (Stoppini, et al. 1991; Bahr, et al. 1995; Zhongrin, et al. 2000). The rats were anesthetized with ketamine (100 mg/kg body weight) and decapitated and the hippocampi were dissected out and sliced into 400 um coronal sections by a McIllwain tissue chopper. Select slices with uninterrupted bright transparent neuronal layers were plated, 1-3 slices/filter, onto Millicell CM filters (Millipore, Bedford, Mass.). For the first 2 days in vitro (DIV) cultures were maintained in 25% horse serum, 50% Basal Media-Eagle (BEM), 25% Eagle's Balanced Salt Solution (EBSS), 25 mM HEPES, 1 mM glutamine, 28 mM glucose, pH 7.2, at 32° C. in a 5% CO₂ humidified atmosphere. The slice cultures were then switched to 25% horse serum, 50% BEM and EBSS modified so that the potassium concentration was 2.66 mM, for another 5 DIV. After 7 DIV, the cultures were maintained in physiological potassium containing 5% horse serum medium at 35° C. for at least 20 days before any treatment was applied. When the slices were treated, the reagents were applied into the culture medium. At different time points, the slices collected with a brush were washed twice in homogenizing buffer (50 mM HEPES, pH 7.0, 10 mM β-merceptoethanol [BME], 1 mM EDTA, 1 mM EGTA, 0.1 mM phenylmethyl sulfonyl fluoride [PMSF], 2.0 mM benzamidine and 2.0 μg/ml each of aprotinin, leupeptin and pepstatin) and homogenized at 4° C. using a Teflon-glass homogenizer. The homogenate was then divided into two parts, one was centrifuged at 16000×g for 15 min and the supernatant was used to assay activities of PP-2A and PP-1. The rest of the homogenate was diluted 1:1 with a phosphatase inhibitor cocktail (20 mM β-glycerophosphate, 2 mM Na₃VO₄ and 100 mM NaF, pH 7.0) and either used for Western blots or centrifuged at 16000×g for 15 min and the resulting supernatant used to determine the kinase activities.

### Protein Phosphatase Assays

Activities of PP-2A and PP-1 were assayed towards [³²P]phosphorylase-a as a substrate as described previously (Gong, et al. 1994). The phosphatase activity was assayed in 20 μl of reaction mixture containing 50 mM Tris, pH 7.0, 10 mM BME, 0.1 mM EDTA, 7.5 mM caffeine, 7.5 ng/μl [³²P]phosphorylase-a and 0.06 mg/ml slice culture extract. The reaction was started by adding ³²P-phosphorylase-a. After incubation for 30 min at 30° C., 7 μl of the reaction mixture was spotted on to 31ETCHR chromatography paper which had been prespotted with 10 μl stop solution (4 mM ATP+20% TCA). Then the ³²P released was separated from the protein-incorporated ³²P by paper chromatography in 5% TCA and 0.2 M NaCl, paper strips dried, cut and counted by Cerenkov radiation. A PP-1 specific inhibitor, inhibitor-1 (Hitken, et al. 1982) was included in the assays for PP-2A activity. PP-1 activity was calculated by subtracting the PP-2A activity from the total phosphorylase-a phosphatase activity (PP-1/PP-2A) assayed in the absence of inhibitor-1.

### Protein Kinase Assays

CaMKII activity was measured in 25 μl of buffer containing 50 mM HEPES, pH 7.5, 10 mM MgCl₂, 2.0 mM CaCl₂, 10 mM BME, 10 μg/ml calmodulin (CaM), 20 μM syntide (Sigma, St Louis Mo., USA), 0.06 mg/ml slice extract and 200 μM [γ³²P] ATP. The reaction was initiated by adding [γ³²P] ATP. After incubation for 10 min at 30° C., 10 μl reaction mixture was removed and spotted on to phosphocellulose membrane. The membrane was then washed five times in 1% phosphoric acid to remove non-protein incorporated ³²P, dried and counted by Cerenkov radiation. The activity of PKA was determined as above except the reaction mixture contained 70 mM NaHPO₄, pH 6.8, 14 mM MgCl₂, 1.4 mM EDTA, 30 μM malantide (Sigma, St Louis, Mo., USA), 200 μM [γ³²P] ATP and 0.06 mg/ml slice extract.

MAPK activity was determined by immunoprecipitating the enzyme from 50 μg of extract with 2 μg of anti-ERK1/2 antibody which recognizes ERK phosphorylated at Thr-185 and Tyr-187. Immobilized protein G (Pierce, Rockford, Ill.), 20 μl, was mixed in 50 mM Tris, pH 7.4, 150 mM NaCl, 10 mM NaF, 1.0 mM Na₃VO₄, 2.0 mM EGTA, 1 mM PMSF, 5 μg/ml leupeptin, 5 μg/ml aprotinin, 2 μg/ml pepstatin, 25 μg/ml phosphoramidon. After incubation at 4° C. overnight, the mixture was centrifuged and the beads were washed three times. The beads were then resuspended in 50 mM Tris, pH 7.4, containing 10 mM MgCl₂. MAPK substrate peptide (UBI, Lake Placid, N.Y.) was employed for the MAPK assay. The bead-bound MAPK was suspended in 20 μl of buffer and incubated at 30° C. for 30 min in the reaction mixture containing 30 mM Tris, pH 7.4, 10 mM MgCl₂, 10 mM NaF, 1.0 mM Na₃VO₄, 2.0 mM EGTA, 10 mM β-mercaptoethanol and 200 μM [γ³²P] ATP.

### Radioimmuno-Dot-Blots and Western Blots

Levels of phosphorylation of tau at different sites were assayed by the radioimmuno-dot-blots of the slice homogenates as described previously (Khatoon, et al. 1992). Triplicate samples of each homogenate were applied to nitrocellulose membrane (Schleicher and Schuell, Keene, N. H.) and dried at 37° C. The primary tau antibodies used were as follows: polyclonal antibodies (pAbs) pS-262 (1:1000) to P-Ser 262 (Bio-source), pS-212 (1:1000) to P-Ser 212 (Bio-source), pS-214 (1:1000) to P-Ser 214 (Bio-source), R145d (1:3000) to P-Ser 422 (Tanaka, et al. 1998), R134d (1:5000) to total tau (Tatebayashi, et al. 1999) or monoclonal antibodies (mAbs) PHF1 (1:200) to P-Ser 396/404 (Greenberg and Davies; Otvos, et al. 1994) and 12E8 (1:500) to P-Ser 262/356 (Seubert, et al. 1995).

Other antibodies useful for the methods of the present invention include but are not limited to: phosphorylation-independent anti-tau antibodies such as monoclonal antibodies (mAb) T46 and T14 (specific to human tau) (Kosik et al., Neuron, 1:817-825, 1988—source, Zymed); rabbit polyclonal antibody 17026 made against the recombinant protein of the longest human tau isoform (Ishihara et al., Neuron, 24:751-762, 1999); and mAb T49 (specific to mouse tau) (Mawal-Dewan et al., J. Biol. Chem., 269:30981-30987, 1994); phosphorylation-dependent anti-tau antibodies such as mAb TI (Binder et al., J. Cell Biol., 101: 1371-1378, 1985; Szendrei et al., J. Neurosci. Res., 34:243-249, 1993); mAb PHF6 (Hoffmann et al., Biochemistry, 36:8114-8124, 1997); mAb AT8 (Goedert et al., Biochem. J., 301:871-877, 1994; Matsuo et al., Neuron, 13:989-1002, 1994—source, Innogenetics, Inc., Ghent, Belgium); mAb AT270 (Goedert et al., 1994, supra; Matsuo et al., 1994, supra—source, Innogenetics, Inc., Ghent, Belgium), and rabbit polyclonal antibodies T3P (Lee et al., Science, 251:675-678, 1991).

The phosphorylation of tau at Ser 262 and the levels of MAP2 and phosphorylated NF-H/M were assayed by ¹²⁵I-Western blots. For tau 10% and for MAP2 and NF-H/M 7.5% SDS-polyacrylamide gel electrophoresis (PAGE), as described originally by Laemmli (Laemmli, et al. 1970) was employed. The protein bands were transferred on to Immobilon-P membrane (Millipore, Bedford, Mass.) and probed with pAb pS-262 (1:1,000, Biosource) or mAb SMI 31 to phosphoneurofilaments-H/M subunits (pNF-H/M), or mAb SMI 52 to MAP2 (Sternberger Monoclonal, Inc.). Both immuno-dot-blots and Western blots were developed with ¹²⁵I-radiolabeled secondary antibodies and radioimmunoreactivity was visualized and quantitated using a phosphorimager (Fujifilm BAS-1500) and TINA 2.0 software (Raytest lsotopenmessgeräte GmbH)

### Immunohistochemistry

After different treatments some of the hippocampal slices were fixed in periodate/lysine/paraformaldehyde solution (Mclean, et al. 1974) at 33° C. for 5 h and then kept in 1% Triton-x-100 in PBS (pH 7.4) for 72 h at room temperature to improve the penetration of the antibodies. The culture slices were then incubated in blocking solution containing PBS, 0.1% TritonX-100 and 10% normal horse serum for 3 h at room temperature. Thereafter, the cultures were rinsed in PBS and incubated for 2 days in primary antibody at 4° C. The primary antibodies used were as follows: mAb SMI 31 to pNF-H/M (1:10,000 Sternberger Monoclonals Incorporated), mAb SMI 52 to MAP2 (1:20,000 Sternberger Monoclonals Incorporated) and pAb pS-262 (1; 1000) to tau phosphorylated at Ser-262. The immunoreactivity was visualized by using peroxidase-conjugated goat antimouse/rabbit IgG (1:1000, Jackson) for 3 h at 37° C. Peroxidase was detected using 0.05% diaminobenzidine (DAB) and H₂O₂ (0.01%) for 10 min.

### Lactate Dehydrogenase (LDH) Activity and Protein

The LDH released into the culture medium from the slices was determined colorimetrically using Cytotox 96R Non-Radioactive Cytotoxicity Assay Kit (Promega, Madison, Wis.) according to the manufacturer's protocol. The assay was carried out in 96-well microplates, and the results were read by a kinetic microplate reader (Molecular Devices) at a wavelength of 490 nm. Protein concentrations were assayed by the modified Lowry method (Bensadoun and Weinstein, 1976).

### EXAMPLE A

### Okadaic Acid (OA) Inhibits PP-2A and Stimulates CaMKII Activity

Since the activity of PP-2A is compromised and is, to date, the only known likely cause of the protein phosphorylation/dephosphorylation imbalance and consequent abnormal hyperphosphorylation of tau and neurofibrillary degeneration in AD brain, we elected to employ for the present study as a model the organotypic culture of adult rat hippocampal slices in which the PP-2A activity was inhibited by OA. The hippocampal slice culture allows direct access to the mammalian brain and the culture can be maintained up to several weeks. This ex vivo system provides a direct and practical access to mammalian brain for studying the effect of pharmacological compounds on the biology of specific proteins and the cascades involved.

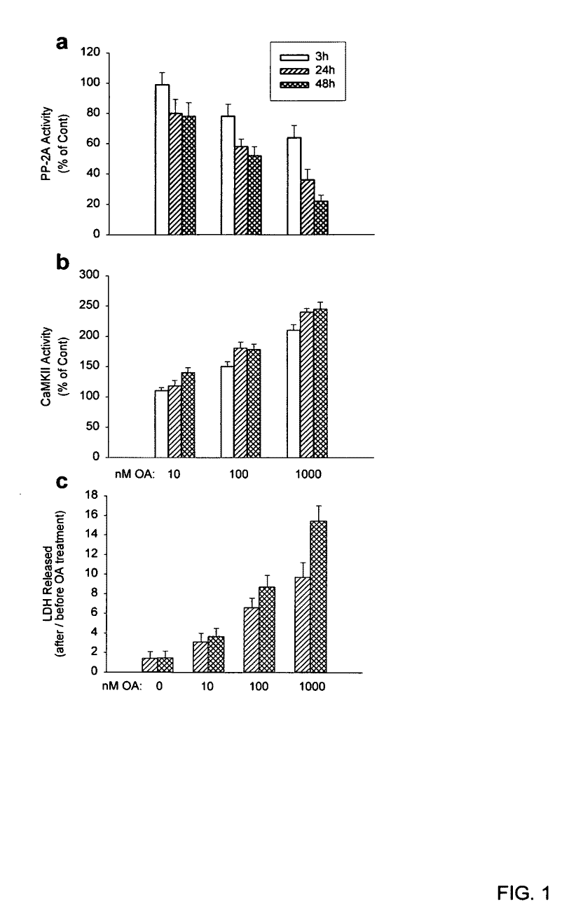

We first investigated the effect of different concentrations of OA for different time periods on the inhibition of PP-2A/PP-1 activities, and consequent stimulation of protein kinases (FIG. 1). We found that 10 nM OA inhibited ˜20% of PP-2A activity during 24 h treatment with no further change up to 48 h treatment studied. OA concentrations of 100 nM and 1 μM resulted in ˜40% and ˜65% inhibition of PP-2A activity, respectively during 24 h treatment. Treatment up to 48 h at either concentration of OA produced only a small additional inhibition of PP-2A activity. However, in agreement with previous studies in which metabolically active rat brain slices were treated with 0.1 to 5 μM OA up to 3 h (Gong, et al. 2000; Bennecib, et al. 2000, 2001) no inhibition of PP-1 activity was detected (Fig. not shown).

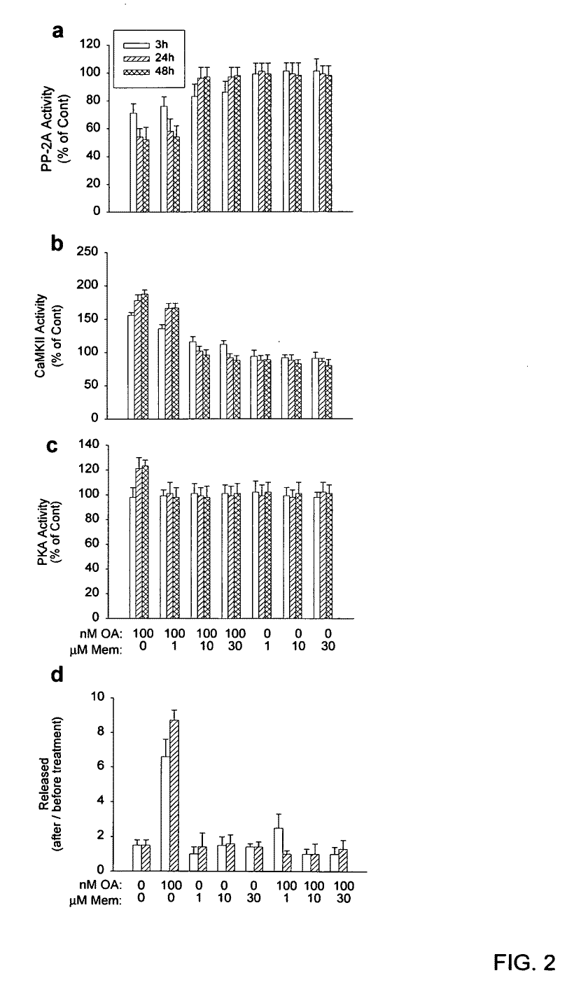

Several protein kinase activities are known to be regulated by reversible phosphorylation and some of these kinases are substrates for PP-2A. We determined the activities of CaMKII, PKA, GSK-3 and cdk5 in the OA-treated and control-treated slice cultures. The CaMKII activity increased with increase in the inhibition of PP-2A activity by OA treatment (FIG. 1b). An increase of ˜20%, ˜70% and ˜140%, respectively was observed in cultures treated with 10, 100 and 1,000 nM OA for 24 h. An increase of ˜20% was observed in PKA activity in the slice cultures treated with 100 nM OA for 24 h or 48 h (FIG. 2c). However, no significant change in the activities of GSK-3 or cdk5 in the OA-treated cultures was detected (Fig. not shown). The cell death in the cultures as determined by assaying LDH activity released in the culture medium (a ratio of after to before OA treatment) was markedly increased both with increase in the OA concentration up to 1 μM and duration of the treatment up to 48 hours studied (FIG. 1 c). To keep any non-specific cytotoxic effects of OA low and to have a model of a significant inhibition of PP-2A activity, we chose the treatment of the slice cultures with 100 nM of the drug for 24 h for all subsequent studies.

### EXAMPLE B

### Memantine Restores the OA-Altered PP-2A and CaMKII Activities to the Normal Level

The activity of CaMKII is stimulated by Ca²⁺/CaM through its autophosphorylation at Thr-286/287 (Miller, et al. 1988) and is regulated by PP-2A which dephosphorylates this site (Bennecib, et al. 2001). Thus, stimulation of CaMKII activity by inhibition of PP-2A provided a very useful non-NMDA pathway model of a protein phosphorylation/dephosphorylation imbalance. Employing this model we investigated the effect of Memantine on the phosphorylation of tau and the protein kinase and protein phosphatase activities involved. The hippocampal slices in culture were treated with 100 nM OA with or without different concentrations of Memantine in the medium for 3-48 h. We found that 10 μM Memantine during 24 h restored the OA-induced changes in the activities of PP-2A, CaMKII and PKA to normal levels (FIG. 2 a-c). Memantine had no significant effect on the activities of cdk5 or GSK-3 in the OA-treated cultures (Fig. not shown), or the activities of PP-2A, CaMKII or PKA in the control/untreated cultures (FIG. 2 a-c). The effect of Memantine on the restoration of PP-2A and CaMKII activities could be observed at 1 μM concentration but the full effect was seen at 10 μM concentration of the drug. Neither increase of Memantine from 10 μM to 30 μM nor duration of the treatment from 24 h to 48 h resulted in any significant additional effect on the restoration of either PP-2A or CaMKII activity. The OA-induced cell death in the cultures was completely inhibited by 10 μM Memantine, and significant effect was observed at as low as 1 μM of the drug studied (FIG. 2 d). In the control cultures Memantine had no effect on the LDH activity in the medium using 1-30 μM concentrations of the drug investigated.

### EXAMPLE C

### Memantine Restores Tau Phosphorylation to Normal Level

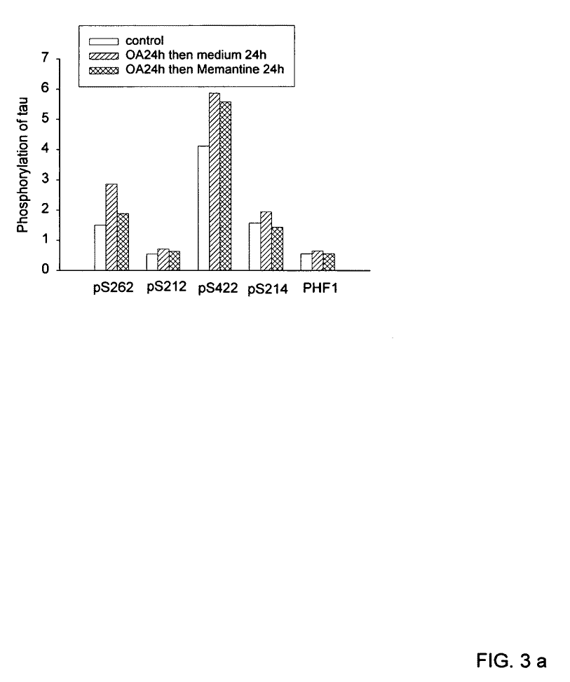

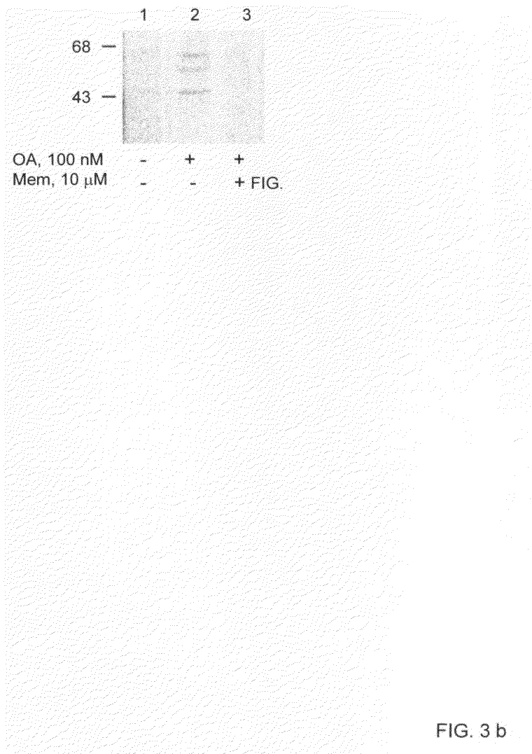

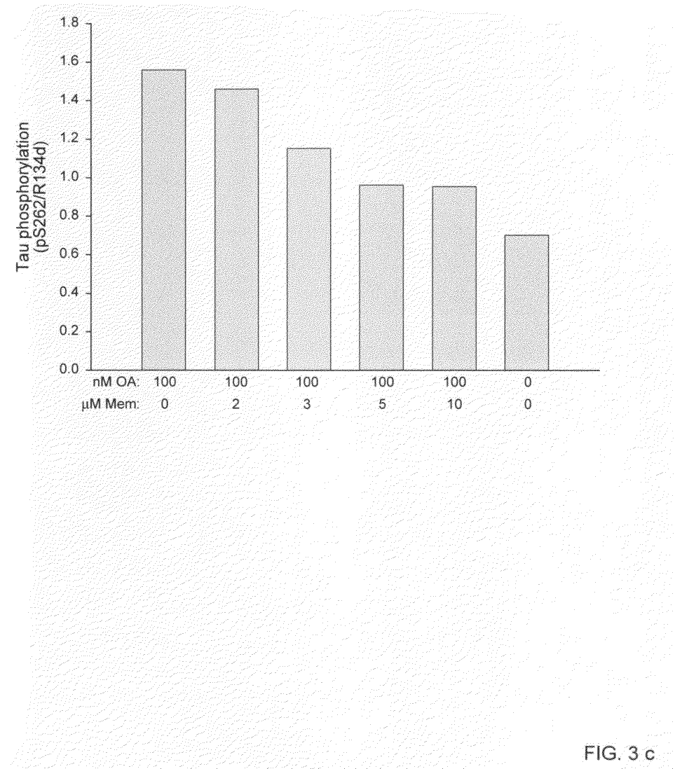

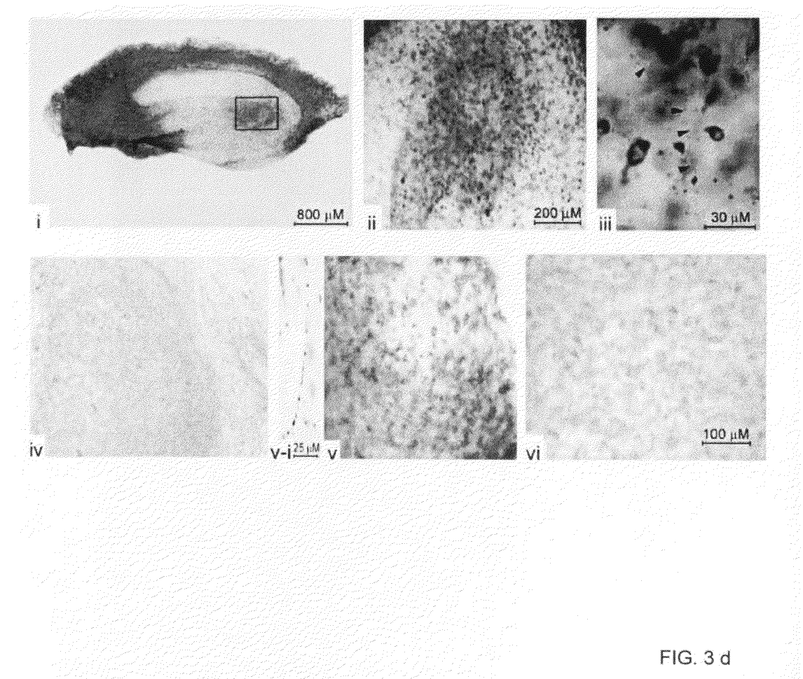

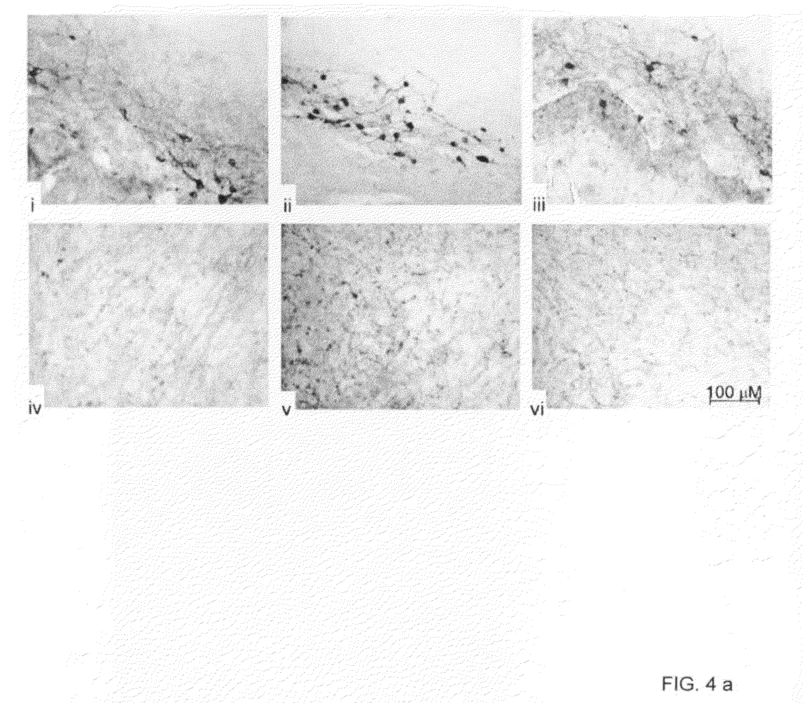

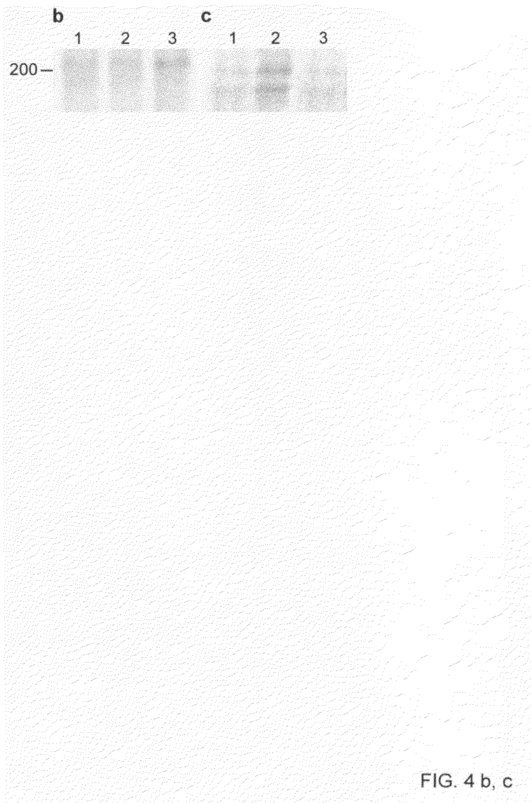

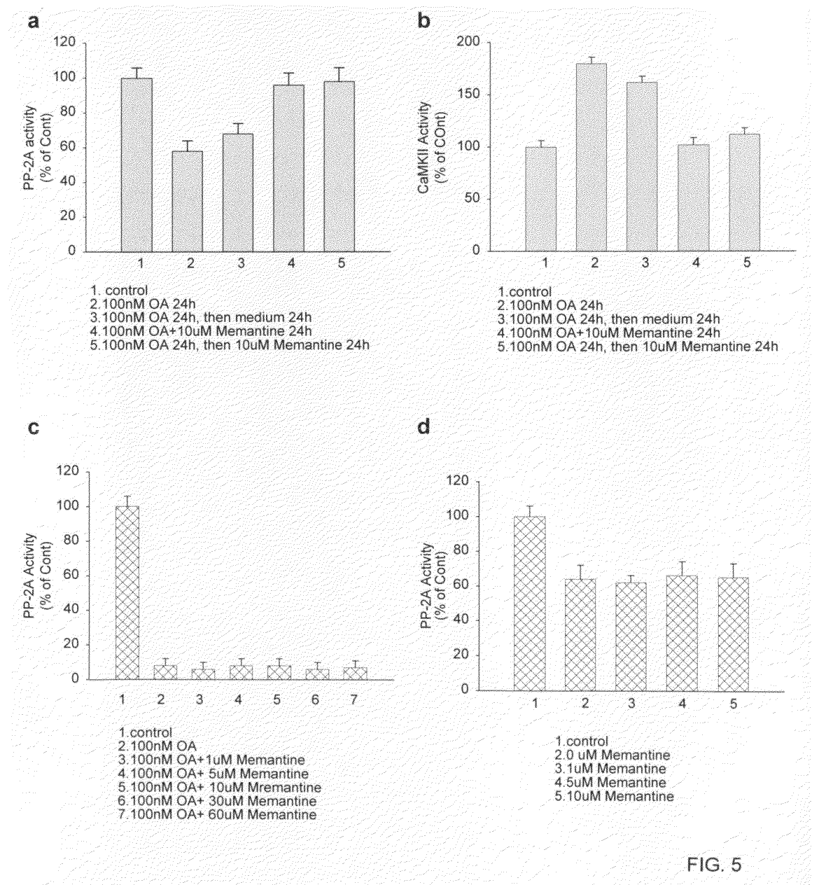

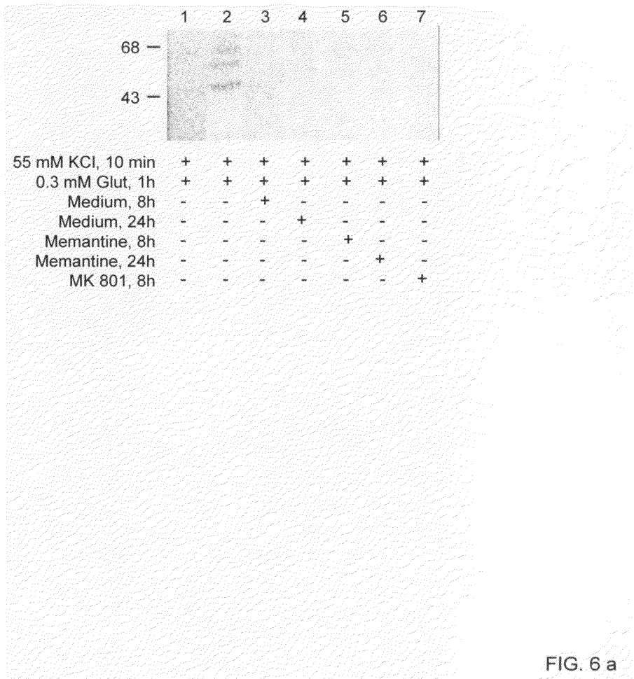

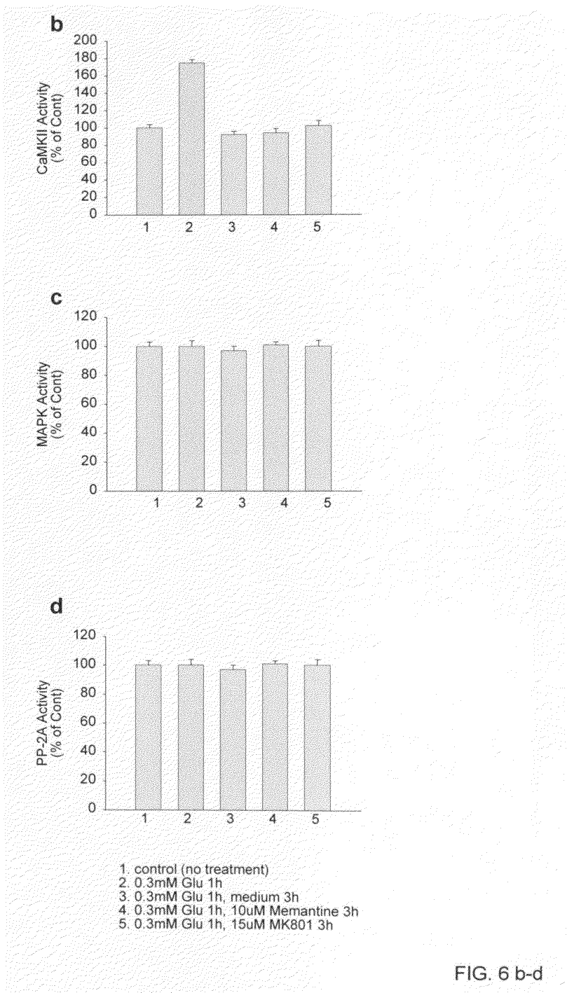

PP-2A downregulates the activity of CaMKII and CaMKII is a major tau Ser-262 kinase in the mammalian brain (Sironi, et al. 1999; Bennecib, et al. 2001). Since we found in the OA-treated hippocampal cultures a marked increase in CaMKII activity and its restoration to normal level by Memantine, we studied in these cultures the effect of these treatments on the phosphorylation of tau at Ser-262 and as a control at Ser-212, Ser-214, Ser-396/404 and Ser-422. Tau Ser-212 is known to be phosphorylated by cdk5 and MAP kinase, Ser-214 by protein kinase A (PKA), Ser-396/404 by GSK-3β and cdk5 and Ser-422 by stress activated protein kinases (Pei, et al., 2001). We determined the levels of total tau in these cultures by [¹²⁵I] radioimmuno-dot-blots using rabbit antibody 134d to tau. Memantine had no detectable effect on the level of total tau in the cultures. A marked increase in the phosphorylation of tau at Ser-262 and Ser-422 and a modest increase at Ser-214 were observed in the OA-treated cultures (FIG. 3a). Further treatment with 10 μM Memantine for 24 h restored the tau phosphorylation at Ser-262 and Ser-214 to normal levels (FIG. 3 a,b). However, Memantine had no effect on the OA-induced phosphorylation of tau at Ser-422 (FIG. 3a).

In order to determine the minimal concentration of Memantine that could restore the phosphorylation of tau at Ser-262 to normal levels, we investigated the effect of 2-10 μM Memantine in the OA-treated cultures by radioimmuno-dot-blot assays. We found that 2 μM Memantine inhibited the tau phosphorylation at Ser-262 and that this effect was maximal at 5 μM concentration of the drug (FIG. 3c).

Immunohistochemical staining of the untreated and treated cultures with phosphodependent rabbit antibody to phospho tau Ser-262 revealed a marked increase in the p-Ser-262 staining in cells in the area corresponding to stratum oriens and alvus in the OA-treated cultures (FIG. 3d). Long processes, presumably axons with irregular contour and short rod-shaped fragments reminiscent of degenerating axons were often seen along the outer regions of stratum radiale (FIG. 3d, v-i). In the cultures treated with 10 μM Memantine for 24 h following the OA treatment, the p-Ser-262 immunostaining of the neurons markedly decreased (FIG. 3d, v-i).

### EXAMPLE D

### Memantine Inhibits Aggregation of MAP2 and Neurofilaments

A protein phosphorylation/dephosphorylation imbalance in the neuron might not only affect the phosphorylation of tau but like in AD, might also affect other cytoskeletal proteins. We studied immunohistochemically the accumulation of MAP2 and pNF-H/M subunits in the OA-treated cultures and the cultures in which the OA treatment was followed by the Memantine treatment. We found that following the OA treatment, the MAP2 immunostaining increased markedly in the somatodendritic compartments of neurons, possibly interneurons, with a corresponding decrease in the neuropil in an area roughly corresponding to stratum oriens (FIG. 4a, i, ii). Dendritic dystrophic fragments with the characteristic of beaded uneven contour, alternating swollen and shrunken segments were seen suggesting a degenerating of the neurons. In the Memantine treated cultures a decrease in the degeneration and restoration of the staining of the neuropil were observed (FIG. 4a, iii). Western blots revealed a decrease in MAP2 in the OA-treated cultures and a reversal to normal levels by treatment with Memantine (FIG. 4 b).

The immunohistochemical labeling of OA treated cultures with antibodies to pNF-H/M also revealed an increase in phosphorylation and accumulation of NF-H/M in the neuronal cell bodies and their neurites in the areas corresponding to stratum oriens and alveus. Thick tortuous, thread-like and beaded fragmented neurites were also abundantly seen in the OA treated cultures (FIG. 4a, v). Memantine, 10 μM, during 24 h treatment partially reversed these pathological changes (FIG. 4a, v). Western blots of the OA- and OA plus Memantine-treated cultures confirmed the reversal of phosphorylation and accumulation of NF-H/M subunits by Memantine (FIG. 4 b).

### EXAMPLE E

### The Restorative Effect of Memantine on the Activities of PP-2A and CaMKII is not by its Direct Interaction with OA

Since Memantine only restored the OA-induced decrease in PP-2A and increase in CaMKII but had no effect on these activities in the control (untreated) cultures, we investigated whether the Memantine effect was due to any direct interaction with OA. For this purpose we treated the hippocampal slices in culture either with 100 nM OA plus 10 μM Memantine or with OA alone for 24 h, followed by a wash and then treatment with or without Memantine for another 24 h. We found that the removal of OA after 24 h treatment restored the PP-2A and CaMKII activities slightly, whereas the treatment of the cultures with both OA and Memantine for 24 h or with OA for 24 h, wash and then with Memantine for 24 h almost completely restored the two enzyme activities (FIG. 5 a,b). These findings suggested that the effect of Memantine on PP-2A and CaMKII activities was unlikely to be through any direct interaction with OA. Furthermore, the addition of OA (100 nM) to a 16,000×g extract of homogenate of untreated cultures inhibited ˜90% of PP-2A activity and the addition of different concentrations of Memantine, 1 μM to 60 μM had no significant effect on the phosphatase activity (FIG. 5 c). Similarly the addition of Memantine, 1-10 μM to the 16,000×g extract of the OA-treated cultures failed to restore the PP-2A activity (FIG. 5 d). All these studies taken together unequivocally demonstrated that Memantine restored PP-2A activity and probably as a consequence the CaMKII activity through some signaling pathway and not by any direct interaction with OA.

### EXAMPLE F

### Effect of Memantine on PP-2A and CaMKII Activities is Unlikely to be Due Only to its Activity as an NMDA Antagonist

CaMKII can be activated either through autophosphorylation induced by okadaic acid or activation of NMDA receptor. In the cultured cells, stimulation of NMDA receptor can lead to reduction of PP-2A activity (Shing, et al. 2001). However, little is known about this relationship in the brain. Therefore, we investigated whether the restorative effect of Memantine could have been as an NMDA receptor antagonist. We used various concentrations of glutamate to treat the cultured slices and at different time intervals examined the changes in PP-2A and CaMKII activities. We found that the treatment of the hippocampal slice cultures with 0.3 mM glutamate for 1 h (FIG. 6 a) but not 24 h (data not shown) produced a marked increase in CaMKII activity. However, this change in CaMKII activity was not accompanied by any changes in the activities of PP-2A or MAP kinase (FIG. 6 b,c). Furthermore, replacement of glutamate from the cultures by fresh medium with or without 10 μM Memantine or 15 μM high affinity NMDA antagonist, MK801 restored the CaMKII activity to normal level and had no effect on the activities of PP-2A or MAP kinase. These studies suggested that the activation of the NMDA receptor by its natural agonist, glutamate activates CaMKII without affecting the PP-2A activity.

### DISCUSSION

In the present study we found ˜75% inhibition of PP-2A and no detectable inhibition of PP-1 with up to 1 μM OA in the rat hippocampal slice cultures during 48 h. In the OA-treated hippocampal slice cultures, a marked increase in the activity of CaMKII and no significant alteration in the activities of PKA, cdk5 and GSK-3β were observed. Associated with these changes in the activities of PP-2A and CaMKII, a dramatic increase in the phosphorylation of tau at Ser-262 and Ser-422 were observed. The hyperphosphorylation of tau at Ser-262 was most likely due to an increase in CaMKII activity as this kinase is the major tau Ser-262 kinase in the mammalian brain (Sironi, et al. 1998; Bennecib, et al. 2001). The phosphorylation of tau at Ser-422 is known to be catalyzed by stress-activated protein kinases (Pei, et al. 2001). The hyperphosphorylation of tau at this site observed in the present study is most likely due to stimulation of the stress-activated protein kinases. The protein phosphorylation/dephosphorylation imbalance and the hyperphosphorylation of tau in the OA-treated hippocampal slice cultures was associated with a several-fold increase in cell death as determined by LDH activity. Thus, OA-treated rat hippocampal slice in culture provided an excellent ex vivo model of AD-type neurofibrillary degeneration in which the effect of pharmacological compounds can be directly tested in adult mammalian hippocampus. Treatment of the OA-treated hippocampal slices in culture with 10 μM Memantine practically completely restored the activities of PP-2A and CaMKII, and phosphorylation of tau at Ser-262 but not of Ser-422 to normal state and inhibited the associated neurodegeneration within 24 h. The restoration of the activities of PP-2A and CaMKII and the inhibition of the OA-induced cell death by Memantine were detectable with as low as 1 μM concentration of the drug studied. The inhibition of the OA-induced abnormal hyperphosphorylation of tau at Ser-262 was detectable using as low as 2 μM Memantine, and the maximal effect was observed at 5 μM of the drug during 24 h. Ser-262 and Ser-422 are known to be major abnormally phosphorylated sites in AD. Ser-262 is the only site abnormally hyperphosphorylated in the microtubule binding domains and the phosphorylation of this site, which is believed to be dynamically involved in tau's activity in stabilizing microtubules, results in inhibition of the microtubule assembly-promoting activity of tau (Biernat, et al. 1993; Singh, et al, 1996). In the present study, the reversal of the OA-induced cell death and the abnormal hyperphosphorylation of tau at Ser-262, but not at Ser-422 to normal-like state by Memantine is consistent with the critical role of the former site in converting tau into an inhibitor/toxic molecule. The phosphorylation of tau at Ser-422 is apparently a later event because this site is phosphorylated in PHF and not cytosolic AD P-tau, and a recent study has confirmed its association to relatively mature tangles in transgenic mice expressing tau P301L mutation (Götz, et al. 2001). The fact that Memantine treatment which completely reversed the PP-2A-induced cell death had no effect on phosphorylation of Ser-422 suggests that this site might not be involved in cytotoxicity but mainly in promoting tau's self assembly into PHF/neurofibrillary tangles.

The immunohistochemical studies revealed abnormal hyperphosphorylation at Ser-262 and accumulation of tau in the OA-treated cultures. The hyperphosphorylation of tau was found primarily in the cells of the stratum oriens and the alveus and in a focal area close to CA3. The cells of this area, some of which might have migrated to this area in culture, showed especially intense immunostaining. Abnormally hyperphosphorylated tau was found to be aggregated in neurites. Treatment of these cultures with Memantine restored in large part the hyperphosphorylation and aggregation of tau to normal-like state during 24 h.

The OA-induced protein phosphorylation/dephosphorylation imbalance not only affected tau but also revealed fragmented MAP2 staining in dendrites and hyperphosphorylation and aggregation of NF-M/H subunits. These changes in the immunostaining of both MAP2 and NF-H/M were also partially reversed by the Memantine treatment. Memantine reversed the hyperphosphorylation of NF-H/M and increased the levels of MAP2, consistent with the inhibition of neurofibrillary degeneration.

The restoration of the OA-induced protein phosphorylation/dephosphorylation imbalance by Memantine was most likely through its effect on PP-2A signaling pathway and neither solely as an NMDA antagonist nor by any direct interaction between OA and Memantine. Memantine, 10 μM, which had no significant effect on the activities of either PP-2A or CaMKII on normal control cultures, restored the activities of both PP-2A and CaMKII and the consequent abnormal hyperphosphorylation of tau both when administered along with OA or after removal of OA from the culture medium. In contrast, in vitro addition of Memantine, 1 μM to 60 μM, to an extract of the cultured slices had no effect on the PP-2A activity inhibited with 100 nM OA. Similarly, Memantine, 1 to 10 μM, had no significant effect in vitro on the PP-2A activity of the extract of hippocampal slices which were cultured in the presence of 100 nM OA for 24 h. These findings demonstrated that Memantine neither had any direct interaction with OA nor it inhibited OA's binding to PP-2A. These in vitro findings also showed the absence of any direct interaction between Memantine and PP-2A.

We found that the treatment of the hippocampal slice cultures with 0.3 mM glutamate resulted in a marked increase in CaMKII activity without any effect on the activities of either PP-2A or MAP kinase, suggesting that the stimulation of glutamate receptors, which include the NMDA receptors, produces an intracellular Ca²⁺ influx which stimulates CaMKII activity, but has no effect on PP-2A activity. Thus, the restoration of the activities of PP-2A and CaMKII and the abnormal hyperphosphorylation of tau to normal-like state by Memantine in the OA-treated hippocampal slice cultures probably involves the modulation of PP-2A signaling, the exact nature of which remains to be understood. It is most likely through this latter effect, that Memantine has a positive therapeutic effect on moderate to severely demented AD patients (Reisberg, et al. 2000).

### Therapeutic or Preventive Examples in Mouse Models of Abnormal Tau Phosphorylation

Early efforts to develop transgenic (Tg) mouse models of tauopathies focused on replicating neuronal tau pathology by overexpressing human tau proteins in neurons which lead to neuronal and axonal degeneration with muscle weaknesses (Ishihara et al., Am. J. Pathol., 158:555-562, 2001). Specifically, in these mice, the longest four-repeat human brain tau isoform is expressed under control of two different neuron-specific promoters (Gotz et al., Ann. NY Acad. Sci., 2000, 920:126-33). In a first model, utilizing the human Thy1 promoter, transgenic tau is hyperphosphorylated and abnormally localized to cell bodies and dendrites. In a second model, which makes use of a human Thy1.2 expression vector, transgenic expression levels are much higher, and an additional phenotype is observed: Large numbers of pathologically enlarged axons containing neurofilament- and tau-immunoreactive spheroids are present, especially in spinal cord. Signs of Wallerian degeneration and neurogenic muscle atrophy are observed. Behaviorally, these transgenic mice show signs of muscle weakness.

Higuchi et al. (Neuron, 35:433-46, 2002) have recently developed another Tg mice overexpressing human tau in both neurons and glia. These mice do not develop neuronal tau inclusions, but they form glial tau pathologies recapitulating those found in human tauopathies.

Prototypical tauopathies are exemplified by frontotemporal dementia with parkinsonism linked to chromosome 17 (FTDP-17). The discovery of tau gene mutations in FTDP-17 kindreds provided unequivocal evidence that tau abnormalities cause neurodegenerative disease (Hutton et al., Nature, 393:702-705, 1998). Intronic and exonic FTDP-17 tau gene mutations cause disease by altering the functions or levels of tau in the CNS (Hong et al., Science, 282:1914-1917, 1998; Hutton et al. 1998, supra).

Tau Tg mice overexpressing human tau with the most common (P301L) FTDP-17 mutation has been produced (Lewis et al., Nat. Genet., 25:402-405, 2000; Götz et al., J. Biol. Chem., 276:529-534, 2001). Expression of human tau P301L results in motor and behavioural deficits in transgenic mice, with age- and gene-dose-dependent development of NFT. This phenotype occurs as early as 6.5 months in hemizygous and 4.5 months in homozygous animals. NFT and Pick-body-like neuronal lesions occur in the amygdala, septal nuclei, pre-optic nuclei, hypothalamus, midbrain, pons, medulla, deep cerebellar nuclei and spinal cord, with tau-immunoreactive pre-tangles in the cortex, hippocampus and basal ganglia. Areas with the most NFT have reactive gliosis. Spinal cord has axonal spheroids, anterior horn cell loss and axonal degeneration in anterior spinal roots. Peripheral neuropathy and skeletal muscle with neurogenic atrophy is also observed. Brain and spinal cord contains insoluble tau that co-migrates with insoluble tau from AD and FTDP-17 brains. The phenotype of mice expressing P301L mutant tau mimics features of human tauopathies and provides, along with mice overexpressing wild-type human tau protein, a good model for investigating the pathogenesis of diseases with NFT.

Genetic deficiency of two ApoE receptors (ApoERs), known as very-low-density lipoprotein receptor (VLDLR) and ApoER2, causes tau hyperphosphorylation that is readily detectable at weaning (Hiesberger et al., Neuron, 24:481-489, 1999). VLDLR and ApoER2 are also receptors for Reelin (Reln), a protein that controls neuronal positioning during brain development (Rice and Curran, Genes Dev., 13:2758-2773, 1999; Gupta et al., Nat. Rev. Genet., 3:342-355, 2002). Mice that are mutant for Rein also have high levels of tau phosphorylation (Hiesberger et al., 1999, supra).

NPC-1 gene mutations cause Niemann-Pick type C(NPC), a neurodegenerative storage disease resulting in premature death in humans. Spontaneous mutation of the NPC-1 gene in mice generates a similar phenotype, usually with death ensuing by 12 weeks of age (Loftus et al., Science 277:232-235, 1997). Both human and murine NPC are characterized neuropathologically by ballooned neurons distended with lipid storage, axonal spheroid formation, demyelination, and widespread neuronal loss. Multiple sites in neurofilaments (NFs), MAP2, and tau are hyperphosphorylated as early as 4 weeks of age and correlate with a significant increase in activity of the cyclin-dependent kinase 5 (cdk5) and accumulation of its more potent activator, p25, a proteolytic fragment of p35 (Bu et al., J. Neurosci. 22:6515-25, 2002).

In the present Example, the concentrations of 1-aminocyclohexane derivative (e.g., memantine or neramexane) resulting in therapeutically meaningful decrease in the abnormal tau hyperphosphorylation in OA ex vivo studies are anticipated to be within the range of 2-5 μM, in any event, different amounts may be tried such as would result in a 45% reduction in phosphorylation at Ser-262, 45% at Ser-212, and 20% at Ser-214, are further tested in various transgenic mouse models of tauopathies described above. Alternatively, according to the present invention, 1-aminocyclohexane derivatives are administered to wild-type mice (or rats) after they have been injected into hippocampus with OA or calyculin A, another potent and specific inhibitor of protein phosphatase (PP)-2A and PP-1 (at the same final intra-brain concentrations as used in ex vivo studies, supra). Specifically, each type of model animals is divided into two groups: a control group, which receives no 1-aminocyclohexane treatment, and an experimental group, which receives the 1-aminocyclohexane derivative (such as memantine or neramexane). Drug administration is carried on over defined periods of time and is followed by testing (using immunodetection methods and enzymatic assays disclosed above), (i) levels of hyperphosphorylated tau which can be measured in CSF fluid by comparing phosphorylated tau to tau levels; (ii) amount of neurofibrillary tangles (NFT) and Pick-body-like neuronal lesions neuropil threads/dystrophic neuritis and loss of synapses; neurofibrillary tangles, Pick bodies, neuropil threads/dystrophic neuritis and loss of synapses are detected by immunohistochemical staining using antibodies to tau, MAP2 and NF-H/M, and in the case of synaptic loss by using cresyl violet and Nissel staining; (iii) CaMKII activity, and (iv) PP-2A and PP-1 activity within various regions/cell types of the brain and spinal cord. The decrease in either of the first three criteria and the improvement in the last criteria in the experimental group (as compared to the control group) is used as a measure of the effectiveness of the 1-aminocyclohexane derivative therapy of the invention. The animal models are further used to determine the optimal dosages, efficacy, toxicity as well as side effects associated with the 1-aminocyclohexane derivative therapy of the invention.

### Pharmaceutical Compositions

The active ingredients of the invention, together with one or more conventional adjuvants, carriers, or diluents, may be placed into the form of pharmaceutical compositions and unit dosages thereof, and in such form may be employed as solids, such as coated or uncoated tablets or filled capsules, or liquids, such as solutions, suspensions, emulsions, elixirs, or capsules filled with the same, all for oral use; in the form of suppositories or capsules for rectal administration or in the form of sterile injectable solutions for parenteral (including intravenous or subcutaneous) use. Such pharmaceutical compositions and unit dosage forms thereof may comprise conventional or new ingredients in conventional or special proportions, with or without additional active compounds or principles, and such unit dosage forms may contain any suitable effective amount of the active ingredient commensurate with the intended daily dosage range to be employed. Tablets containing twenty (20) to one hundred (100) milligrams of active ingredient or, more broadly, ten (10) to two hundred fifty (250) milligrams per tablet, are accordingly suitable representative unit dosage forms.

### EXAMPLES OF REPRESENTATIVE PHARMACEUTICAL COMPOSITIONS

With the aid of commonly used solvents, auxiliary agents and carriers, the reaction products can be processed into tablets, coated tablets, capsules, drip solutions, suppositories, injection and infusion preparations, and the like and can be therapeutically applied by the oral, rectal, parenteral, and additional routes. Representative pharmaceutical compositions follow.

(a) Tablets suitable for oral administration which contain the active ingredient may be prepared by conventional tabletting techniques.

(b) For suppositories, any usual suppository base may be employed for incorporation thereinto by usual procedure of the active ingredient, such as a polyethyleneglycol which is a solid at normal room temperature but which melts at or about body temperature.

(c) For parenteral (including intravenous and subcutaneous) sterile solutions, the active ingredient together with conventional ingredients in usual amounts are employed, such as for example sodium chloride and double-distilled water q.s., according to conventional procedure, such as filtration, aseptic filling into ampoules or IV-drip bottles, and autoclaving for sterility.

Other suitable pharmaceutical compositions will be immediately apparent to one skilled in the art.

The following examples are again given by way of illustration only and are not to be construed as limiting.

### EXAMPLE 1

### Tablet Formulation

A suitable formulation for a tablet containing 10 milligrams of active ingredient is as follows:

| | Mg. |
|---|---|
| Active Ingredient | 10 |
| Lactose | 63 |
| Microcrystalline Cellulose | 21 |
| Talcum | 4 |
| Magnesium stearate | 1 |
| Colloidal silicon dioxide | 1 |

### EXAMPLE 2

### Tablet Formulation

Another suitable formulation for a tablet containing 100 mg is as follows:

| | Mg. |
|---|---|
| Active Ingredient | 100 |
| Potato starch | 20 |
| Polyvinylpyrrolidone | 10 |
| Film coated and colored. | |

The film coating material consists of:

| | |
|---|---|
| Lactose | 100 |
| Microcryst. Cellulose | 80 |
| Gelatin | 10 |
| Polyvinylpyrrolidone, crosslinked | 10 |
| Talcum | 10 |
| Magnesium stearate | 2 |
| Colloidal silicon dioxide | 3 |
| Color pigments | 5 |

### EXAMPLE 3

### Capsule Formulation

A suitable formulation for a capsule containing 50 milligrams of active ingredient is as follows:

| | Mg. |
|---|---|
| Active Ingredient | 50 |
| Corn starch | 20 |
| Dibasic calcium phosphate | 50 |
| Talcum | 2 |
| Colloidal silicon dioxide | 2 |

filled in a gelatin capsule.

### EXAMPLE 4

### Solution for Injection

A suitable formulation for an injectable solution containing one percent of active ingredient is as follows:

| | |
|---|---|
| Active Ingredient mg | 12 |
| Sodium chloride mg | 8 |
| Sterile water to make ml | 1 |

### EXAMPLE 5

### Liquid Oral Formulation

A suitable formulation for 1 liter of a liquid mixture containing 2 milligrams of active ingredient in one milliliter of the mixture is as follows:

| | G. |
|---|---|
| Active Ingredient | 2 |
| Saccharose | 250 |
| Glucose | 300 |
| Sorbitol | 150 |
| Orange flavor | 10 |
| Sunset yellow. | |
| Purified water to make a total | |
| of 1000 ml. | |

### EXAMPLE 6

### Liquid Oral Formulation

Another suitable formulation for 1 liter of a liquid mixture containing 20 milligrams of active ingredient in one milliliter of the mixture is as follows:

| | G. |
|---|---|
| Active Ingredient | 20.00 |
| Tragacanth | 7.00 |
| Glycerol | 50.00 |
| Saccharose | 400.00 |
| Methylparaben | 0.50 |
| Propylparaben | 0.05 |
| Black currant-flavor | 10.00 |
| Soluble Red color | 0.02 |
| Purified water to make a total | |
| of 1000 ml. | |

### EXAMPLE 7

### Liquid Oral Formulation

Another suitable formulation for 1 liter of a liquid mixture containing 2 milligrams of active ingredient in one milliliter of the mixture is as follows:

| | G. |
|---|---|
| Active Ingredient | 2 |
| Saccharose | 400 |
| Bitter orange peel tincture | 20 |
| Sweet orange peel tincture | 15 |
| Purified water to make a total | |
| of 1000 ml. | |

### EXAMPLE 8

### Aerosol Formulation

180 g aerosol solution contain:

| | G. |
|---|---|
| Active Ingredient | 10 |
| Oleic acid | 5 |
| Ethanol | 81 |
| Purified Water | 9 |
| Tetrafluoroethane | 75 |

15 ml of the solution are filled into aluminum aerosol cans, capped with a dosing valve, purged with 3.0 bar.

### EXAMPLE 9

### TDS Formulation

100 g solution contain:

| | G. |
|---|---|
| Active Ingredient | 10.0 |
| Ethanol | 57.5 |
| Propyleneglycol | 7.5 |
| Dimethylsulfoxide | 5.0 |
| Hydroxyethylcellulose | 0.4 |
| Purified water | 19.6 |

1.8 ml of the solution are placed on a fleece covered by an adhesive backing foil. The system is closed by a protective liner which will be removed before use.

### EXAMPLE 10

### Nanoparticle Formulation

10 g of polybutylcyanoacrylate nanoparticles contain:

| | G. |
|---|---|
| Active Ingredient | 1.00 |
| Poloxamer | 0.10 |
| Butylcyanoacrylate | 8.75 |
| Mannitol | 0.10 |
| Sodiumchloride | 0.05 |

Polybutylcyanoacrylate nanoparticles are prepared by emulsion polymerization in a water/0.1 N HCl/ethanol mixture as polymerization medium. The nanoparticles in the suspension are finally lyophilized under vacuum.

It is to be understood that the invention is not to be limited to the exact details of operation, or to the exact compositions, methods, procedures, or embodiments shown and described, as obvious modifications and equivalents will be apparent to one skilled in the art, and the invention is therefore to be limited only by the full scope which can be legally accorded to the appended claims.

### REFERENCE

1. Aitken A, Bilham T and Cohen P Eur J Biochem 1982(126) 235-246
2. Alger, B. E. Dhanjal, S. S., Dingledine, R. Garthwaite, J. Henderson, G. (1984) Brain Slices, P P, 381-437 Plenum Press, NY
3. Alonso, A. del C., Grundke-Iqbal, I. and Iqbal, K. Nature Med. 2:783-787, 1996
4. Alonso. A. Del C., Zaidi, T., Grundke-Iqbal, 1. and Iqbal, K., (1994) Proc Natl Acad Sci USA 91, 5562-5566
5. Alonso, A. del C., Grundke-Iqbal, I., Barra, H. S and Iqbal, K. (1997) Proc Natl Acad Sci USA 94, 298-303
6. B. A. Bahr Long-term hippocampal slice—A model System for investigating synaptic Mechanisms and Pathologic Processes J Neurosci Research 1995(42): 294-305
7. Baumann K., Mandelkow E. M., Biernat J. Et al (1993) Abnormal Alzheimer-like phosphorylation of tau-protein by cyclin-dependent kinase cdk2 and cdk5 FEBS Lett 336, 417-424
8. Bennecib, M. Gong C-X, Grundke-Iqbal, 1. And Iqbal, K. Role of protein phosphatase-2A and -1 in the regulation of GSK-3, cdk5 and cdc2 and the phosphorylation of tau in rat forebrain FEBS Lett 485 (2000) 87-93
9. Bennecib M., Gong C-X, Wegiel, J., Lee M. H., Grundke-Iqbal, I. And Iqbal, K. (2000) Inhibition of protein phosphatase and regulation of tau phosphorylation in rat brain Alzheimer's Report 3, 56 295-304
10. Bennecib, M., Gong C-X, Grundke-Iqbal, 1. and Iqbal, K. (2001) Inhibition of PP-2A upregulates CaMKII in rat forebrain and induces hyperphosphorylation of tau at Ser262/396 FEBS Lett 490, 15-22
11. Bensadoun A. and Weinstein D. (1976) Anal Biochem 70, 241-250
12. Berridge M. J., Lipp P. And Bootman M. D. (2000) The versatility and universality of calcium signaling Nat Rev Mol Cell Biol 1, 11-21
13. Bialojan, C., and Takai, A. (1988) Biochem. J. 256, 283-290.
14. Biernat, J., Gusteke, N., Drewes, G., Mandelkow, E.-M., and Mandelkow, E. (1993) Neuron 11:153-163.
15. Colbran R. J. (1993) J. Biol Chem 268, 7163-7170
16. Danysa, W., Parsons, C. G., Kornhnber, J., Schmidt, W., Quack, G. (1997) Aminoadamantances as NMDA receptor antagonists and antiparkinsonian agents Preclinical studies Neurosci Biobehav Rev 21, 455-468
17. Drewes G., Lichtenberg-Kraay B., Doring F., et al (1992) Mitogen activated protein (MAP) kinase transforms tau protein into an Alzheimer-like state EMBO J 11, 2131-2138
18. Drewes, G., Trinczek, B., Illenberger, S., Biernat, J., Schmitt-Ulms, G. Et al Microtubule-associated protein/microtubule affinity-regulating kinase (p110 mark): A novel protein kinase that regulates tau microtubule interaction and dynamic instability by phosphorylation at the Alzheimer specific site Ser262 J Biol Chem 270, 7679-7688
19. Goedert M., Cohen E. S., Jakes R. And Cohen P. (1992) p42 MAP kinase phosphorylation sites in microtubule-associated protein tau are dephosphorylated by protein phosphatase 2A Implications for Alzheimer's disease FEBS Lett 312, 95-99
20. Goedert M., Hasegawa, M., Jakes, R. Et al (1997) Phosphorylation of microtubule-associated protein tau by stree-activated protein kinase FEBS Lett 409, 57-62
21. Gong C-X, Singh T. J., Grundke-Iqbal, I and Iqbal, K. (1993) Phosphoprotein phosphatase activities in Alzheimer disease brain J Neurochem 61, 921-927
22. Gong C-X, Grundke-Iqbal, I. and Iqbal, K. (1994a) Dephosphorylation of Alzheimer's disease abnormally phosphorylated tau by protein phosphatase-2A Neurosci 61, 765-772
23. Gong C-X, Grundke-Iqbal, I., Damuni Z and Iqbal, K. (1994b) Dephosphorylation of microtubule associated protein tau by protein phosphatase-1 and -2C and its implication in Alzheimer disease FEBS Lett 341, 94-98
24. Gong C-X, Singh T. J., Grundke-Iqbal, I. And Iqbal, K. (1994c) Alzheimer's disease abnormally phosphorylated tau is dephosphorylated by protein phosphatase-2B (calcineurin) J Neurochem 62, 803-806
25. Gong C-X, Shaikh, S. Wang J-Z et al (1995) Phosphatase activity toward abnormally phosphorylated tau: decrease in Alzheimer disease brain J Neurochem 65, 732-738
26. Gong C-X, Lidsky T., Wegiel J. Et al (2000) Phosphorylation of microtubule-associated protein tau is regulated by protein phosphatase 2A in mammalian brain—Implication for neurofibrillary degeneration in Alzheimer's disease J Biol Chem 275, 5535-5544
27. Gong C-X, Wegiel, J., Lidsky, T., Zuck, L., Avila, J., Wisniewski, H. M., Iqbal-Grundke, I., and Iqbal K. (2000) Brain Res 853, 229-309
28. Gong C-X, Wu Q-L, Wang J-Z, Iqbal, K. and Grundke-Iqbal, I. Unpublished
29. Grundke-Iqbal, I., Iqbal, K., Quinlan, M., Tung, Y-C., Zaidi, T. and Wisniewski, H. M. (1986) J Biol Chem, 261, 6084-6089
30. Hu, et al. 2002. Am. J. Pathol
31. I. Grundke-Iqbal I., Iqbal, K., Quinlan, M., Tung, Y-C., Wisniewski, H. M. and Binder, L. I. (1986) Proc Natl Acad Sci USA 83, 4913-4917
32. Iqbal, K., Grundke-Iqbal, I., Zaidi, T., Merz, P. A., Wen, G. Y., Shaikh, S. S., Wisniewski, H. M., Alafuzoff, I. and Winblad, B. Lancet, 2, 421-426, 1986.
33. Iqbal, K., Grundke-Iqbal, I., Smith, A. J., George, I., Tung, Y-C and Zaidi, T. (1989) Proc Natl Sci USA 86, 5646-5650
34. Jicha, G. A., Weaver, C. Lane, E., Vianna, C. (1999) J Neurosci 19, 7486-7494
35. Kornhuber, J., Bormann, J., Retz, W., Zlubers, M., Riederer, P. (1989) Memantine displace [3H] MK801 at therapeutic concentrations in postmortem human frontal cortex Eur J Pharmacol 166, 589-590
36. Laabich, A., Li, G. And Cooper, N. G. Calcium/calmodulin-dependent protein Kinase II containing a nuclear localizing signal is altered in retinal neurons exposed to N-methyl-D-aspartate Brain Res Mol Brain Res 76:253-265 (2000)
37. Laabich, A. and Cooper N. G. F. Regulation of Calcium/calmodulin-dependent protein kinase II in the adult retina is mediated by Ionotropic glutamate receptor Exp Eye Res (1999) 68, 703-713
38. Laemmli, U. K. (1970) Nature 227, 680-685
39. Lee, V. M.-Y., Balin, B. J., Otvos, L. J. and Trojanowski, J. Q. (1991) Science 251, 675-678
40. Lindwall, G., and Cole, R. D. (1984) J Biol Chem 259, 5301-5305
41. Lipton, P., Aitken, P. G., Dudek, F. E., Eskessen, K. Et al (1995) J Neurosci Methods 59, 151-156
42. Litersky J. M., Johnson G. V., Jakes R. Et al (1996) Tau protein is phosphorylated by cyclic AMP-dependent protein kinase and calcium/calmodulin-dependent protein kinase II within its microtubule-binding domains at Ser-262 and Ser356 Biochem J 316, 655-660
43. Mattson M. P., Laferla, F. M., Chan S. L., Leissring M. Calcium signaling in the ER: its role in neuronal plasticity and neurodegenerative disorders Trends Neurosci 23, 222-229
44. Mclean, I. W. and Nakne P. K. (1974) J Histochem Cytochem 22, 1077-1083
45. Miller, S. G., Patton, B. L. and Kennedy M. B. (1988) Neuron 1, 593-604
46. Morishima-Kawashima, M., Hasegawa, M., Takio, K., Suzuki, M. (1995) Proline-directed and non-proline-directed phosphorylation of PHF-tau J Biol Chem 270, 823-829
47. Muller, W. E., Mutschler, E., Riederer, P. 1995 Noncompetitive NMDA receptor antagonist with fast open-channel blocking kinetics and strong voltage-dependency as potential therapeutic agents for Alzheimer's dementia Pharmacopsychiatry 28, 113-124
48. Otvos, L., Feiner, L., Long, E., Szendrei, G., Goedert, M. And Lee, V. M. Y. (1994) J Neurosci Res 39, 669-673
49. Parsons C. G., Danysz, W., Quack, G. (1999) Memantine is a clinically well tolerated NMDA receptor antagonist—a review of preclinical data Neuropharmacology 38, 735-767
50. Paudel, H. K., Lew, J., Ali, Z. And Wang J. H. (1993) I Biol Chem 268, 23512-23518
51. Reynolds C. H., Utlon, M. A., Gibb G. M. et al (1997) Stress-activated protein kinase/c-jun N-terminal kinase phosphorylates tau protein I Neurochem 68, 1736-1744
52. Seubert, P., Mawal-Dewan, M., Barbour, R., Jakes, R., Goedert, M., Johnson, G. V. W., Litersky, J. M., Schenk, D., Lieberburg, I., Trojanowski, J. Q. and Lee, V. M. Y. (1995) J Biol Chem 270, 18917-18922
53. Shing F. C. and Nikolaus J. S. An NMDA receptor signaling complex with protein phosphatase 2A J Neurosci 15, 2001 21(20) 7985-7992
54. Singh, T. J., Haque, N., Grundke-Iqbal, I. and Iqbal, K. (1995) Rapid Alzheimer-like phosphorylation of tau by the synergistic actions of non-proline-dependent protein kinases and GSK-3 FEBS Lett 387, 145-148
55. Singh, T. J., Grundke-Iqbal, I. And Iqbal, K. (1995) J Neurochem 64, 1420-1423
56. Singh T. J., Wang J-Z, Novak M et al (1996) Calcium/calmodulin-dependent protein kinase II phosphorylates tau at Ser 262 but only partially inhibits its binding to microtubules FEBS Lett 387, 145-148
57. Sironi J., Yen S. H., Gondal J. A. et al (1998) Ser 262 in human recombinant tau protein is a markedly more favorable site for phosphorylation by CaMKII than PKA Or PhK FEBS Lett 436, 471-475
58. L. Stoppini, P-A. Buchs and D. Muller A simple method for organotypic culture of nervous tissue J Neurosci Methods 37 (1991) 173-182
59. Tanaka T., Zhong J., Iqbal, K. Et al (1998) The regulation of phosphorylation of tau in SY5Y neuroblastoma cells: the role of protein phosphatase FEBS Lett 426, 248-254
60. Wang J-Z, Wu Q-L, Smith A. et al (1998) Tau is phosphorylated by GSK-3 at several sites found in Alzheimer disease and its biological activity markedly inhibited only after it is prephosphorylated by A kinase FEBS Lett 436, 28-34
61. Wang J-Z, Tung Y-C, Wang Y-P, Li X-T, Iqbal, K., and Iqbal Grundke. (2001) FEBS
62. Winblad B. and Poritis, N. Memantine in severe dementia: results of the 9M-Best study (benefit and efficacy in severely demented patients during treatment with memantine) Int J Geriatr Psychiatry 1999 Feb., 14(2) 135-146
63. Yoshimura, Y., Sogawa, Y. And Yamauchi, T. (1999) FEBS Lett 446, 239-242.

We claim:

1. A method for treatment of a condition resulting from hyperphosphorylation of microtubule protein tau selected from parkinsonism-dementia, argyrophilic grain dementia, British type amyloid angiopathy, corticobasal degeneration, dementia pugilistica, autism with self-injury behavior, Down's syndrome, frontotemporal dementia with parkinsonism linked to chromosome 17, Gerstmann-Straussler-Scheinker disease, Hallenvorden-Spatz disease, inclusion body myositis, multiple system atrophy, myotonic dystrophy, Niemann-Pick type C neurodegenerative storage disease, presenile dementia, prion protein cerebral amyloid angiopathy, progressive supranuclear palsy, progressive subcortical gliosis, post-encephalitic parkinsonism, subacute sclerosing panencephalitis, tangle only dementia, spasticity, AIDS dementia, neuropathic pain, cerebral ischemia, epilepsy, glaucoma, hepatic encephalopathy, multiple sclerosis, stroke, tardive dyskinesia, drug tolerance, opiate/alcohol dependence, thermal hyperalgesia, mechanical allodynia, malaria, Borna virus, and Hepatitis C, such method comprising the step of administering, to a patient in need thereof, an effective amount of a compound selected from those of formula I 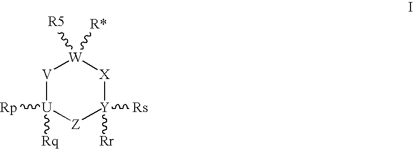
wherein:
R* is -(A)ₙ-(CR¹R²)ₘ—NR³R⁴,
n+m=0, 1, or 2,
A is selected from linear or branched lower alkyl (C₁-C₆), linear or branched lower alkenyl (C₂-C₆), and linear or branched lower alkynyl (C₂-C₆),
R¹ and R² are independently selected from hydrogen, linear or branched lower alkyl (C₁-C₆), linear or branched lower alkenyl (C₂-C₆), and linear or branched lower alkynyl (C₂-C₆),
R³ and R⁴ are independently selected from hydrogen, linear or branched lower alkyl (C₁-C₆), linear or branched lower alkenyl (C₂-C₆), and linear or branched lower alkynyl (C₂-C₆), or together form alkylene (C₂-C₁₀) or alkenylene (C₂-C₁₀) or together with the N form a 3-7-membered azacycloalkane or azacycloalkene, including substituted (alkyl (C₁-C₆), alkenyl (C₂-C₆)) 3-7-membered azacycloalkane or azacycloalkene,
Rₚ, R_{q}, Rᵣ, and Rₛ are independently selected from hydrogen, linear or branched lower alkyl (C₁-C₆), linear or branched lower alkenyl (C₂-C₆), linear or branched lower alkynyl (C₂-C₆), cycloalkyl (C₃-C₆) and phenyl, and one of Rₚ and R_{q} and one of Rᵣ and Rₛ combine together to represent a lower alkylene —(CH₂)ₓ— bridge wherein x is 2-5, inclusive, which alkylene bridge, in turn, combines with R⁵ to form an additional lower alkylene —(CH₂)_{y}— bridge, wherein y is 1-3, inclusive,
U-V-W-X-Y-Z is selected from
cyclohexane,
cyclohex-2-ene,
cyclohex-3-ene,
cyclohex-1,4-diene,
cyclohex-1,5-diene,
cyclohex-2,4-diene, and
cyclohex-2,5-diene,
and its optical isomers and pharmaceutically-acceptable acid or base addition salts thereof.

2. The method of claim 1, wherein the compound of formula I is selected from:
1-amino adamantane,
1-amino-3-phenyl adamantane,
1-amino-methyl-adamantane,
1-amino-3,5-dimethyl adamantane,
1-amino-3-ethyl adamantane,
1-amino-3-isopropyl adamantane,
1-amino-3-n-butyl adamantane,
1-amino-3,5-diethyl adamantane,
1-amino-3,5-diisopropyl adamantane,
1-amino-3,5-di-n-butyl adamantane,
1-amino-3-methyl-5-ethyl adamantane,
1-N-methylamino-3,5-dimethyl adamantane,
1-N-ethylamino-3,5-dimethyl adamantane,
1-N-isopropyl-amino-3,5-dimethyl adamantane,
1-N,N-dimethyl-amino-3,5-dimethyl adamantane,
1-N-methyl-N-isopropyl-amino-3-methyl-5-ethyl adamantane,
1-amino-3-butyl-5-phenyl adamantane,
1-amino-3-pentyl adamantane,
1-amino-3,5-dipentyl adamantane,
1-amino-3-pentyl-5-hexyl adamantane,
1-amino-3-pentyl-5-cyclohexyl adamantane,
1-amino-3-pentyl-5-phenyl adamantane,
1-amino-3-hexyl adamantane,
1-amino-3,5-dihexyl adamantane,
1-amino-3-hexyl-5-cyclohexyl adamantane,
1-amino-3-hexyl-5-phenyl adamantane,
1-amino-3-cyclohexyl adamantane,
1-amino-3,5-dicyclohexyl adamantane,
1-amino-3-cyclohexyl-5-phenyl adamantane,
1-amino-3,5-diphenyl adamantane,
1-amino-3,5,7-trimethyl adamantane,
1-amino-3,5-dimethyl-7-ethyl adamantane,
1-amino-3,5-diethyl-7-methyl adamantane,
1-amino-3-methyl-5-propyl adamantane,
1-amino-3-methyl-5-butyl adamantane,
1-amino-3-methyl-5-pentyl adamantane,
1-amino-3-methyl-5-hexyl adamantane,
1-amino-3-methyl-5-cyclohexyl adamantane,
1-amino-3-methyl-5-phenyl adamantane,
1-amino-3-ethyl-5-propyl adamantane,
1-amino-3-ethyl-5-butyl adamantane,
1-amino-3-ethyl-5-pentyl adamantane,
1-amino-3-ethyl-5-hexyl adamantane,
1-amino-3-ethyl-5-cyclohexyl adamantane,
1-amino-3-ethyl-5-phenyl adamantane,
1-amino-3-propyl-5-butyl adamantane,
1-amino-3-propyl-5-pentyl adamantane,
1-amino-3-propyl-5-hexyl adamantane,
1-amino-3-propyl-5-cyclohexyl adamantane,
1-amino-3-propyl-5-phenyl adamantane,
1-amino-3-butyl-5-pentyl adamantane,
1-amino-3-butyl-5-hexyl adamantane,
1-amino-3-butyl-5-cyclohexyl adamantane,
and their acid addition compounds.

3. The method of claim 1, wherein the compound of formula I is memantine.

4. The method of claim 1, wherein the condition is selected from: frontotemporal dementia with parkinsonism linked to chromosome 17 (FTDP-17), corticobasal degeneration (CBD), progressive supranuclear palsy (PSP), progressive subcortical gliosis (PSG), Niemann-Pick type C(NPC) neurodegenerative storage disease, and Argyrophilic Grain disease, and the compound of formula I is memantine.

5. The method of claim 3, wherein memantine is administered in the amount of 5 to 200 mg/kg.
